(19) United States
(12) Patent Application Publication
(10) Pub. No.: US 2019/0292259 A1
(43) Pub. Date: Sep. 26, 2019

(54) METHODS AND PHARMACEUTICAL COMPOSITIONS FOR THE TREATMENT OF NON SMALL CELL LUNG CANCER (NSCLC) THAT COEXISTS WITH CHRONIC OBSTRUCTIVE PULMONARY DISEASE (COPD)

(71) Applicants: INSERM (INSTITUT NATIONAL DE LA SANTE ET DE LA RECHERCHE MEDICALE), Paris (FR); MEDIMMUNE, LLC, Gaithersburg, MD (US); UNIVERSITE PARIS DESCARTES, Paris (FR); UNIVERSITE PIERRE ET MARIE CURIE(PARIS 6), Paris (FR); UNIVERSITE PARIS DIDEROT - PARIS 7, Paris (FR); SORBONNE UNIVERSITE, Paris (FR)

(72) Inventors: Diane DAMOTTE, Paris (FR); Ronald HERBST, Gaithersburg, MD (US); Jerome BITON, Paris (FR); Claudie BANTSIMBA MALANDA, Paris Cedex 06 (FR)

(21) Appl. No.: 16/301,633

(22) PCT Filed: May 24, 2017
(86) PCT No.: PCT/EP2017/062625

§ 371 Date: Nov. 14, 2018

(30) Foreign Application Priority Data
May 24, 2016 (EP) 16305597.3

(51) Int. Cl. C07K 16/28; G01N 33/569; G01N 33/574

(52) U.S. Cl. CPC C07K 16/2818; G01N 33/57423; G01N 33/56966; C07K 16/2827

(57) ABSTRACT

The disclosure relates to methods and pharmaceutical compositions for the treatment of non-small cell lung cancer (NSCLC) that coexists with chronic obstructive pulmonary disease (COPD). In particular, the disclosure relates to a method of treating non-small cell lung cancer (NSCLC) in a patient suffering from chronic obstructive pulmonary disease (COPD) comprising administering to the patient a therapeutically effective amount of an immune checkpoint inhibitor such as is a multispecific antibody comprising at least one binding site that specifically binds to a PD-1 molecule and at least one binding site that specifically binds to a TIM-3 molecule.

### FIELD OF THE INVENTION

The present invention relates to methods and pharmaceutical compositions for the treatment of non-small cell lung cancer (NSCLC) that coexists with chronic obstructive pulmonary disease (COPD).

### BACKGROUND OF THE INVENTION

An increasing body of research has demonstrated that solid tumors represent a complex ecosystem composed of malignant, stromal and endothelial cells, as well as immune cells. Inside this network, the central role of the immune system has been emphasized by a strong association between T cell infiltrate and patients' clinical outcome (1-6). Indeed, in the majority of cancers, a high density of CD8⁺ tumor-infiltrating T lymphocytes (CD8 TILs), together with a T helper cell 1 (Th1) and cytotoxic signature, are associated with a favorable clinical outcome in terms of local tumor spreading, disease free survival, and overall survival (7-9). In some tumors, immune cell aggregates exhibiting an organizational architecture similar to that of canonical secondary lymphoid organs were also observed (10). These tertiary lymphoid structures (TLS) are composed of a T cell zone, in which T cells form clusters with mature dendritic cells (DCs), adjacent to a B cell follicle, characterized by proliferating germinal center B cells and a network of follicular DCs (11). The presence of TLS within tumor tissues was associated with a better clinical outcome in several cancer types, including melanoma and breast cancer (12, 13). In non-small cell lung cancer (NSCLC), high densities of both TLS and CD8 TILs identified a group of patients with the best overall survival, suggesting that TLS may orchestrate in situ the generation of a finely organized adaptive anti-tumor immune response (14). Finally, the major role of immune cells in the tumor microenvironment has been recently highlighted by the success of therapies targeting immune checkpoint molecules, such as cytotoxic T lymphocyte-associated antigen-4 (CTLA-4), programmed cell death-1 (PD-1) and programmed cell death ligand-1 (PD-L1) (15-21). Unfortunately, these new treatments are not efficient in all patients and for all tumor types, reinforcing the need to find new therapeutic targets and also to identify factors predicting clinical response to this kind of immunotherapy.

Within the tumor microenvironment immune cells may also have a dark side linked to the maintenance of a deleterious chronic inflammation. Different types of triggers, including microbial infections and also autoimmune disorders are able to promote cancer development by inducing the establishment of a chronic inflammation. Indeed, it has been estimated that 15-20% of all deaths from cancer worldwide are linked to underlying infections or chronic inflammatory responses (22). For instance, infectious organisms, such as Helicobacter pylori, papillomavirus and hepatitis B or C viruses were respectively demonstrated as involved in the development of gastric cancer (23, 24), cervical cancer (25) and hepatocellular carcinoma (26). Also, patients with inflammatory bowel disease (IBD) or with chronic pancreatitis were shown to have an increased risk of developing colorectal cancer (CRC) (27, 28) and pancreatic cancer (29), respectively, thus strengthening the relationship between inflammation and cancer. In addition, non-steroidal anti-inflammatory drugs have the capacity to reduce the risk of developing certain types of tumor, such as CRC and ovarian cancer (30). Inflammation may favor tumorigenesis by inducing extracellular matrix disruption and DNA damage through the production of pro-inflammatory cytokines, reactive oxygen species and matrix metalloproteinases (MMPs) (31). Once tumors progress to malignancy, pro-inflammatory cytokines, mainly produced by macrophages, may stimulate angiogenesis, promote acquisition of new mutations, and enhance tumor cell migration and invasion (32).

In the lung, chronic obstructive pulmonary disease (COPD) that represents the third leading cause of death worldwide (33), is a pathology characterized by chronic inflammation, and has been recognized as an important risk factor for lung cancer (34). COPD is a heterogeneous pathology, comprised of two major components, obstructive bronchitis (small airways disease) and emphysema (parenchyma destruction). COPD is linked to an abnormal inflammatory immune response of the lung to noxious particle or gazes, and shares its main risk factor, tobacco, with lung cancer (35). The characteristics of COPD⁺ patients, when compared with smokers without COPD, is that they develop more pronounced destructive inflammation of the lung, starting with a strong release of TNF-α and CXCL-8 by epithelial cells and alveolar macrophages, leading to the recruitment of inflammatory monocytes and neutrophils (36). This results in extracellular matrix destruction and cell death through the release of matrix metalloproteinases (MMPs) and reactive oxygen species, ultimately resulting in emphysema (36). This chronic inflammation of the lung, accompanied by increased oxidant and noxious stress can lead to DNA adduct formation, and has logically been suggested to promote the earliest stages of carcinogenesis in COPD⁺ patients (36). Accordingly, the risk of lung cancer development in smokers with COPD is higher than the one in smokers without COPD (37). Genes involved in cell proliferation and survival, such as NF-κB and STAT3, that are up regulated in COPD (38) and activated by pro-inflammatory cytokines, including IL-6 and TNF-α, play probably a major role in this process.

Lung cancer is associated with COPD in approximately 40-60% of the cases. COPD worsens the survival of patients with early-stage NSCLC (39) and emphysema is associated with increased lung cancer mortality, even among patients who have never been active smokers (40). The exact mechanisms involved, including the role of the immune system, are not completely known.

### SUMMARY OF THE INVENTION

The present invention relates to methods and pharmaceutical compositions for the treatment of non-small cell lung cancer (NSCLC) that coexists with chronic obstructive pulmonary disease (COPD). In particular, the present invention is defined by the claims.

### DETAILED DESCRIPTION OF THE INVENTION

In various cancer types, chronic inflammation has been shown to promote carcinogenesis and to hamper the efficiency of tumor immune surveillance. However, the potential impact of chronic obstructive pulmonary disease (COPD), a chronic inflammatory condition of the lung, on the immune contexture of non-small cell lung cancer (NSCLC) is not known. The inventors have deciphered the immune microenvironment in NSCLC according to coexisting COPD. In-depth immune profiling of lung tumor tissues, using immunohistochemical, gene expression and flow cytometry analyses, reveals that COPD disrupts the immune microenvironment of NSCLC. None of the four tumor-infiltrating immune cell populations studied, particularly CD8 tumor-infiltrating lymphocytes (CD8 TILs), were associated with a significant prognostic value in COPD+ patients in contrast to what is observed in non-COPD patients. In accordance, the inventors also determined that CD8 TIL exhaustion, characterized by the co-expression of PD-1/TIM-3 and strictly correlated to the density of CD8 T cells in the tumor nests, was differentially orchestrated in COPD+ NSCLC patients. Indeed, in tumors strongly infiltrated by adaptive immune cells, CD8 TIL exhaustion was higher in moderate to severe COPD patients. A coexisting COPD is associated with a complete loss of CD8 T cell-associated favorable clinical outcome, through a higher proportion of CD8 T cells co-expressing PD-1 and TIM-3 in highly CD8 T cell-infiltrated tumors. In full agreement, the deleterious effect of PD-L1 expression by tumor cells on NSCLC patients' survival was restricted to highly CD8 T cell infiltrated tumors of COPD+ patients. Remarkably, data obtained on 34 advanced-stage NSCLC patients are in favor of a higher efficacy of nivolumab in COPD+ patients. The results suggest that a coexisting COPD, recognized as an important risk factor for lung cancer, is associated with an increased sensibility of TILs to mechanisms developed by malignant cells to avoid tumor immune surveillance and may indicate a higher sensitivity to immune checkpoint blockade therapies.

Accordingly, the a first object of the present invention relates to a method of treating non-small cell lung cancer (NSCLC) in a patient suffering from chronic obstructive pulmonary disease (COPD) comprising administering to the patient a therapeutically effective amount of an immune checkpoint inhibitor.

As used herein, the term “non-small cell lung cancer” or “NSCLC” has its general meaning in the art and includes a disease in which malignant cancer cells form in the tissues of the lung. Examples of non-small cell lung cancers include, but are not limited to, squamous cell carcinoma, large cell carcinoma, and adenocarcinoma.

As used herein, the term “chronic obstructive pulmonary disease” or “COPD” has its general meaning in the art and refers to a set of physiological symptoms including chronic cough, expectoration, exertional dyspnea and a significant, progressive reduction in airflow that may or may not be partly reversible. COPD is a disease characterized by a progressive airflow limitation caused by an abnormal inflammatory reaction to the chronic inhalation of particles. The Global Initiative for Chronic Obstructive Lung Disease (GOLD) has classified 4 different stages of COPD (Table A). In some embodiments, the patient suffers from moderate COPD. In some embodiments, the patient suffers from a severe or very severe COPD.

TABLE A
Gold classification: The volume in a one-second forced exhalation is called
the forced expiratory volume in one second (FEV1), measured in liters. The total
exhaled breath is called the forced vital capacity (FVC), also measured in liters.
In people with normal lung function, FEV1 is at least 70% of FVC.

| | | | |
|---|---|---|---|
| Stage I | Mild COPD | FEV1/FVC < 0.70 | FEV₁ ≥ 80% normal |
| Stage II | Moderate COPD | FEV1/FVC < 0.70 | FEV₁ 50-79% normal |
| Stage III | Severe COPD | FEV1/FVC < 0.70 | FEV₁ 30-49% normal |
| Stage IV | Very Severe COPD | FEV1/FVC < 0.70 | FEV₁ < 30% normal, or <50% |
| | | | normal with chronic |
| | | | respiratory failure present |

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the present invention relates to a method for the prophylactic treatment of non-small lung cancer in a patient suffering from chronic obstructive pulmonary disease comprising administering to the patient a therapeutically effective amount of an immune checkpoint inhibitor.

As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. As used herein, the term “immune checkpoint inhibitor” has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. In particular, the immune checkpoint inhibitor of the present invention is administered for enhancing the proliferation, migration, persistence and/or cytoxic activity of CD8+ T cells in the subject and in particular the tumor-infiltrating of CD8+ T cells of the subject.

In some embodiments, the immune checkpoint inhibitor is a TIM-3 inhibitor.

In some embodiments, the immune checkpoint inhibitor is a PD-1 inhibitor.

As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9.

As used herein, the term “PD-1” has its general meaning in the art and refers to programmed cell death protein 1 (also known as CD279). PD-1 acts as an immune checkpoint, which upon binding of one of its ligands, PD-L1 or PD-L2, inhibits the activation of T cells. Accordingly, the term “PD-1 inhibitor” as used herein refers to a compound, substance or composition that can inhibit the function of PD-1. For example, the inhibitor can inhibit the expression or activity of PD-1, modulate or block the PD-1 signaling pathway and/or block the binding of PD-1 to PD-L1 or PD-L2.

In some embodiments, the immune checkpoint inhibitor is an antibody selected from the group consisting of anti-PD1 antibodies, anti-PDL1 antibodies, anti-PDL2 antibodies, anti-Galectin 9 antibodies and anti-TIM-3 antibodies. In further embodiments, the immune checkpoint inhibitor is an antibody selected from the group consisting of nivolumab (anti-PD-1), pembrolizumab (anti-PD-1) and durvalumab (anti-PD-L1)

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404,097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388.

As used herein, the term “specificity” refers to the ability of an antibody to detectably bind an epitope presented on an antigen (e.g. TIM-3, PD-1, galectin-9, PD-L1 or PD-L2), while having relatively little detectable reactivity with other proteins or structures (such as other proteins presented on CD8 T cells, or on other cell types). Specificity can be relatively determined by binding or competitive binding assays, using, e.g., Biacore instruments, as described elsewhere herein. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules (in this case the specific antigen is a polypeptide). The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

In some embodiments, the antibody comprises human heavy chain constant regions sequences but will induce antibody dependent cellular cytotoxicity (ADCC). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. No. 6,194,551 by ldusogie et al.

In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3.

Antibodies having specificity for TIM-3 are well known in the art and typically include those described in WO2011155607, WO2013006490 and WO2010117057.

Antibodies having specificity for PD-1 or PDL-1 are well known in the art and typically include those described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In some embodiments, the PD-1 inhibitors include anti-PD-L1 antibodies. In some embodiments the PD-1 inhibitors include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224 is a fusion protein of B7-DC; an antibody Fc portion; BMS-936559 (MDX-1105-01) for PD-L1 (B7-H1) blockade.

In some embodiments, the immune checkpoint inhibitor is a multispecific antibody comprising at least one binding site that specifically binds to a PD-1 molecule, and at least one binding site that specifically binds to a TIM-3 molecule.

Multispecific antibodies are typically described in WO2011159877. According to the invention the multispecific antibody of the present invention binds to PD-1 and TIM-3 and inhibits the ability of PD-1 to, for example, bind PD-L1, and inhibits the ability of TIM-3 to, for example, bind galectin-9. Exemplary formats for the multispecific antibody molecules of the present invention include, but are not limited to (i) two antibodies cross-linked by chemical heteroconjugation, one with a specificity to TIM-3 and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies. In some embodiments, the bispecific antibody is obtained or obtainable via a controlled Fab-arm exchange, typically using DuoBody technology. In vitro methods for producing bispecific antibodies by controlled Fab-arm exchange have been described in WO2008119353 and WO 2011131746 (both by Genmab A/S). In one exemplary method, described in WO 2008119353, a bispecific antibody is formed by “Fab-arm” or “half-molecule” exchange (swapping of a heavy chain and attached light chain) between two monospecific antibodies, both comprising IgG4-like CH3 regions, upon incubation under reducing conditions. The resulting product is a bispecific antibody having two Fab arms which may comprise different sequences. In another exemplary method, described in WO 2011131746, bispecific antibodies of the present invention are prepared by a method comprising the following steps, wherein at least one of the first and second antibodies is a antibody of the present invention: a) providing a first antibody comprising an Fc region of an immunoglobulin, said Fc region comprising a first CH3 region; b) providing a second antibody comprising an Fc region of an immunoglobulin, said Fc region comprising a second CH3 region; wherein the sequences of said first and second CH3 regions are different and are such that the heterodimeric interaction between said first and second CH3 regions is stronger than each of the homodimeric interactions of said first and second CH3 regions; c) incubating said first antibody together with said second antibody under reducing conditions; and d) obtaining said bispecific antibody, wherein the first antibody is a antibody of the present invention and the second antibody has a different binding specificity, or vice versa. The reducing conditions may, for example, be provided by adding a reducing agent, e.g. selected from 2-mercaptoethylamine, dithiothreitol and tris(2-carboxyethyl)phosphine. Step d) may further comprise restoring the conditions to become non-reducing or less reducing, for example by removal of a reducing agent, e.g. by desalting. Preferably, the sequences of the first and second CH3 regions are different, comprising only a few, fairly conservative, asymmetrical mutations, such that the heterodimeric interaction between said first and second CH3 regions is stronger than each of the homodimeric interactions of said first and second CH3 regions. More details on these interactions and how they can be achieved are provided in WO 2011131746, which is hereby incorporated by reference in its entirety.

In some embodiments, the immune checkpoint inhibitor is a polypeptide comprising a functional equivalent of TIM-3 or PD-1. As used herein, a “functional equivalent of TIM-3 or PD-1” is a polypeptide which is capable of binding to a TIM-3 or PD-1 ligand, thereby preventing its interaction with TIM-3 or PD-1. The term “functional equivalent” includes fragments, mutants, and muteins of TIM-3 or PD-1. The term “functionally equivalent” thus includes any equivalent of TIM-3 or PD-1 obtained by altering the amino acid sequence, for example by one or more amino acid deletions, substitutions or additions such that the protein analogue retains the ability to bind to a ligand of TIM-3 or PD-1. Amino acid substitutions may be made, for example, by point mutation of the DNA encoding the amino acid sequence. Functional equivalents include molecules that bind a ligand of TIM-3 or PD-1 and comprise all or a portion of the extracellular domains of TIM-3 or PD-1 so as to form a soluble receptor that is capable to trap the ligand of TIM-3 or PD-1. Thus the functional equivalents include soluble forms of the TIM-3 or PD-1. A suitable soluble form of these proteins, or functional equivalents thereof, might comprise, for example, a truncated form of the protein from which the transmembrane domain has been removed by chemical, proteolytic or recombinant methods. Typically, the functional equivalent is at least 80% homologous to the corresponding protein. In a preferred embodiment, the functional equivalent is at least 90% homologous as assessed by any conventional analysis algorithm. The term “a functionally equivalent fragment” as used herein also may mean any fragment or assembly of fragments of TIM-3 or PD-1 that binds to a ligand of TIM-3 or PD-1. Accordingly the present invention provides a polypeptide capable of inhibiting binding of TIM-3 or PD-1 to a ligand of TIM-3 or PD-1, which polypeptide comprises consecutive amino acids having a sequence which corresponds to the sequence of at least a portion of an extracellular domain of TIM-3 or PD-1, which portion binds to a ligand of TIM-3 or PD-1. In some embodiments, the polypeptide corresponds to an extracellular domain of TIM-3 or PD-1.

In some embodiments, the polypeptide comprises a functional equivalent of TIM-3 or PD-1 which is fused to an immunoglobulin constant domain (Fc region) to form an immunoadhesin. Immunoadhesins can possess many of the valuable chemical and biological properties of human antibodies. Since immunoadhesins can be constructed from a human protein sequence with a desired specificity linked to an appropriate human immunoglobulin hinge and constant domain (Fc) sequence, the binding specificity of interest can be achieved using entirely human components. Such immunoadhesins are minimally immunogenic to the patient, and are safe for chronic or repeated use. In some embodiments, the Fc region is a native sequence Fc region. In some embodiments, the Fc region is a variant Fc region. In still another embodiment, the Fc region is a functional Fc region. As used herein, the term “Fc region” is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The adhesion portion and the immunoglobulin sequence portion of the immunoadhesin may be linked by a minimal linker. The immunoglobulin sequence typically, but not necessarily, is an immunoglobulin constant domain. The immunoglobulin moiety in the chimeras of the present invention may be obtained from IgG1, IgG2, IgG3 or IgG4 subtypes, IgA, IgE, IgD or IgM, but typically IgG1 or IgG3. In some embodiments, the functional equivalent of the TIM-3 or PD-1 and the immunoglobulin sequence portion of the immunoadhesin are linked by a minimal linker. As used herein, the term “linker” refers to a sequence of at least one amino acid that links the polypeptide of the invention and the immunoglobulin sequence portion. Such a linker may be useful to prevent steric hindrances. In some embodiments, the linker has 4; 5; 6; 7; 8; 9; 10; 11; 12; 13; 14; 15; 16; 17; 18; 19; 20; 21; 22; 23; 24; 25; 26; 27; 28; 29; 30 amino acid residues. However, the upper limit is not critical but is chosen for reasons of convenience regarding e.g. biopharmaceutical production of such polypeptides. The linker sequence may be a naturally occurring sequence or a non-naturally occurring sequence. If used for therapeutical purposes, the linker is typically non-immunogenic in the subject to which the immunoadhesin is administered. One useful group of linker sequences are linkers derived from the hinge region of heavy chain antibodies as described in WO 96/34103 and WO 94/04678. Other examples are poly-alanine linker sequences.

In some embodiments, the immune checkpoint inhibitor is an inhibitor of TIM-3 or PD-1 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of TIM-3 or PD-1 mRNA by binding thereto and thus preventing protein translation or increasing mRNA degradation, thus decreasing the level of TIM-3 or PD-1, and thus activity, in a cell. For example, antisense oligonucleotides of at least about 15 bases and complementary to unique regions of the mRNA transcript sequence encoding TIM-3 or PD-1 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. TIM-3 or PD-1 gene expression can be reduced by contacting a subject or cell with a small double stranded RNA (dsRNA), or a vector or construct causing the production of a small double stranded RNA, such that TIM-3 or PD-1 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing TIM-3 or PD-1. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

A further object of the present invention relates to a method of modifying the activation status of lung immune cells in a patient suffering from a non-small cell lung cancer (NSCLC) which coexists with chronic obstructive pulmonary disease (COPD) comprising: (i) identifying the patient as a patient suffering from a non-small cell lung cancer (NSCLC) which coexists with chronic obstructive pulmonary disease (COPD); and (ii) administering to said patient a therapeutically effective amount of an immune checkpoint inhibitor.

A further object of the present invention relates to a method of modulating lymphocyte distribution in the tumor microenvironment of a patient suffering from a non-small cell lung cancer (NSCLC) which coexists with chronic obstructive pulmonary disease (COPD) patient comprising: (i) identifying the patient as a patient suffering from a non-small cell lung cancer (NSCLC) which coexists with chronic obstructive pulmonary disease (COPD); and (ii) administering to said patient a therapeutically effective amount of an immune checkpoint inhibitor.

A further object of the present invention relates to a method of preventing TIL exhaustion in the tumor microenvironment of a patient suffering from a non-small cell lung cancer (NSCLC) which coexists with chronic obstructive pulmonary disease (COPD) comprising: (i) identifying the patient as a patient suffering from a non-small cell lung cancer (NSCLC) which coexists with chronic obstructive pulmonary disease (COPD); and (ii) administering to said patient a therapeutically effective amount of an immune checkpoint inhibitor.

A further object refers to a method of predicting whether a patient suffering from a non-small cell lung cancer (NSCLC) will achieve a response with an immune checkpoint inhibitor comprising i) determining whether the patient has a coexisting chronic obstructive pulmonary disease (COPD) and ii) concluding that the patient has a high probability to achieve a response with an immune checkpoint inhibitor when the patients has a coexisting chronic obstructive pulmonary disease.

A further object refers to a method of treating a patient suffering from non-small cell lung cancer (NSCLC), wherein said patient has been pre-determined to have coexisting chronic obstructive pulmonary disease (COPD) and wherein said patient has a high probability to achieve a response with an immune checkpoint inhibitor when the patients has a coexisting chronic obstructive pulmonary disease.

As used herein, the expression “high probability to achieve a response with an immune checkpoint inhibitor” is understood to mean the situation where the patient shows at 50%, at least 60%, at least 70%, at least 80%, at least 90%, at least 95%, least 100% of chance to achieve a response. 50% of chance to achieve a response means that the subject has 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; 100% of chance to achieve a response.

The method is thus particularly suitable for discriminating responder from non-responder. As used herein the term “responder” in the context of the present disclosure refers to a patient that will achieve a response, i.e. a patient where the non-small cell lung cancer is eradicated, reduced or improved. According to the invention, the responders have an objective response and therefore the term does not encompass patients having a stabilized cancer such that the disease is not progressing after the treatment with the immune checkpoint inhibitor. A non-responder or refractory patient includes patients for whom the non-small lung cancer does not show reduction or improvement after the treatment with the immune checkpoint inhibitor. According to the invention the term “non-responder” also includes patients having a stabilized cancer. Typically, the characterization of the patient as a responder or non-responder can be performed by reference to a standard or a training set. The standard may be the profile of a patient who is known to be a responder or non-responder or alternatively may be a numerical value. Such predetermined standards may be provided in any suitable form, such as a printed list or diagram, computer software program, or other media.

In some embodiments, the present invention method of predicting whether a patient suffering from a non-small cell lung cancer (NSCLC) will achieve a response with a TIM-3 inhibitor comprising i) determining whether the patient has a coexisting chronic obstructive pulmonary disease (COPD) and ii) concluding that the patient has a high probability to achieve a response with a TIM-3 inhibitor when the patient has a coexisting chronic obstructive pulmonary disease.

In some embodiments, the present invention method of predicting whether a patient suffering from a non-small cell lung cancer (NSCLC) will achieve a response with a PD-1 inhibitor comprising i) determining whether the patient has a coexisting chronic obstructive pulmonary disease (COPD) and ii) concluding that the patient has a high probability to achieve a response with a PD-1 inhibitor when the patient has a coexisting chronic obstructive pulmonary disease.

A further object of the present invention relates to a method of treating a patient suffering from a non-small cell lung cancer (NSCLC) that coexists with chronic obstructive pulmonary disease (COPD) comprising i) determining the expression level of PD-L1 in a tumor tissue sample obtained from the patient ii) comparing the expression level determined at step i) with a predetermined reference value and iii) administering to the patient a therapeutically effective amount of a PD-1 inhibitor when the level determined at step i) is higher than the predetermined reference value.

As used herein, the term “tumor tissue sample” has its general meaning in the art and encompasses pieces or slices of tissue that have been removed including following a surgical tumor resection. The tumor tissue sample can be subjected to a variety of well-known post-collection preparative and storage techniques (e.g., fixation, storage, freezing, etc.) prior to determining the cell densities. Typically the tumor tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (using an IHC automate such as BenchMark® XT, for obtaining stained slides). The tumour tissue sample can be used in microarrays, called as tissue microarrays (TMAs). TMA consists of paraffin blocks in which up to 1000 separate tissue cores are assembled in array fashion to allow multiplex histological analysis. This technology allows rapid visualization of molecular targets in tissue specimens at a time, either at the DNA, RNA or protein level. TMA technology is described in WO2004000992, U.S. Pat. No. 8,068,988, Olli et al 2001 Human Molecular Genetics, Tzankov et al 2005, Elsevier; Kononen et al 1198; Nature Medicine.

Typically, the expression of PD-L1 in the tumor tissue sample is determined by any well-known method in the art.

In some embodiments, the expression of PD-L1 in the tumor tissue sample is determined by immunohistochemistry. For example, the determination is performed by contacting the tumor tissue sample with a binding partner (e.g. an antibody) specific PD-L1.

Immunohistochemistry typically includes the following steps i) fixing the tumor tissue sample with formalin, ii) embedding said tumor tissue sample in paraffin, iii) cutting said tumor tissue sample into sections for staining, iv) incubating said sections with the binding partner specific for the immune checkpoint protein of interest, v) rinsing said sections, vi) incubating said section with a secondary antibody typically biotinylated and vii) revealing the antigen-antibody complex typically with avidin-biotin-peroxidase complex. Accordingly, the tumor tissue sample is firstly incubated with the binding partners having for the immune checkpoint protein of interest. After washing, the labeled antibodies that are bound to the immune checkpoint protein of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems.

For example, one or more labels can be attached to the antibody, thereby permitting detection of the target protein (i.e the immune checkpoint protein). Exemplary labels include radioactive isotopes, fluorophores, ligands, chemiluminescent agents, enzymes, and combinations thereof. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. ³H, ¹⁴C, ³²P, ³⁵S or ¹²⁵I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In some embodiments, the label is a quantum dot. For example, Quantum dots (Qdots) are becoming increasingly useful in a growing list of applications including immunohistochemistry, flow cytometry, and plate-based assays, and may therefore be used in conjunction with this invention. Qdot nanocrystals have unique optical properties including an extremely bright signal for sensitivity and quantitation; high photostability for imaging and analysis. A single excitation source is needed, and a growing range of conjugates makes them useful in a wide range of cell-based applications. Qdot Bioconjugates are characterized by quantum yields comparable to the brightest traditional dyes available. Additionally, these quantum dot-based fluorophores absorb 10-1000 times more light than traditional dyes. The emission from the underlying Qdot quantum dots is narrow and symmetric which means overlap with other colors is minimized, resulting in minimal bleed through into adjacent detection channels and attenuated crosstalk, in spite of the fact that many more colors can be used simultaneously. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled.

In some embodiments, the resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biomarker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the immune checkpoint protein in the sample, or the absolute number of cells positive for the maker of interest, or the surface of cells positive for the maker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantify the presence of the specified biomarker (i.e. immune checkpoint protein). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms and tissue recognition pattern (e.g. Aperio Spectrum Software, Automated QUantitatative Analysis platform (AQUA® platform), or Tribvn with Ilastic and Calopix software), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biomarker (i.e. the immune checkpoint protein) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. For example, the amount can be quantified as an absolute number of cells positive for the maker of interest. In some embodiments, a score is given to the sample that is a numerical representation of the intensity or amount of the histochemical staining of the sample, and represents the amount of target biomarker (e.g., the immune checkpoint protein) present in the sample. Optical density or percentage area values can be given a scaled score, for example on an integer scale.

Thus, in some embodiments, the method of the present invention comprises the steps consisting in i) providing one or more immunostained slices of tissue section obtained by an automated slide-staining system by using a binding partner capable of selectively interacting with PDL-1, ii) proceeding to digitalisation of the slides of step i).by high resolution scan capture, iii) detecting the slice of tissue section on the digital picture iv) providing a size reference grid with uniformly distributed units having a same surface, said grid being adapted to the size of the tissue section to be analyzed, and v) detecting, quantifying and measuring intensity or the absolute number of stained cells in each unit.

In some embodiments, the method further comprises determining the expression of at least one further immune checkpoint protein. In particular, the method further comprises determining the expression level of TIM-3.

Multiplex tissue analysis techniques are particularly useful for quantifying several immune checkpoint proteins in the tumor tissue sample. Such techniques should permit at least five, or at least ten or more biomarkers to be measured from a single tumor tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.).

In some embodiments, the L-IHC method can be performed on any of a variety of tissue samples, whether fresh or preserved. The samples included core needle biopsies that were routinely fixed in 10% normal buffered formalin and processed in the pathology department. Standard five μιη thick tissue sections were cut from the tissue blocks onto charged slides that were used for L-IHC. Thus, L-IHC enables testing of multiple markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10μιη thick coated polymer backbone with 0.4μιη diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present methods utilize Multiplex Tissue Imprinting (MTI) technology for measuring biomarkers, wherein the method conserves precious biopsy tissue by allowing multiple biomarkers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.). That technique is described in U.S. Pat. No. 7,473,532.

In some embodiments, the method of the present invention utilized the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the patient, ii) TMA samples are then stained with anti-antibodies having specificity of the immune checkpoint protein(s) of interest, iii) the TMA slide is further stained with an epithelial cell marker to assist in automated segmentation of tumour and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

In some embodiments, the expression level of PDL-1 is determined by determining the quantity of mRNA encoding for PD-L1. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In some embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Puhlication No. 2003/0165951 as well as PCT Puhlication No. 99/26299 (published May 27, 1999). Separate populations of semiconductor nanocrystals can he produced that are identifiable based on their different spectral characteristics. For example, semiconductor nanocrystals can he produced that emit light of different colors based on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.). Additional labels include, for example, radioisotopes (such as 3H), metal chelates such as DOTA and DPTA chelates of radioactive or paramagnetic metal ions like Gd3+, and liposomes. Detectable labels that can be used with nucleic acid molecules also include enzymes, for example horseradish peroxidase, alkaline phosphatase, acid phosphatase, glucose oxidase, beta-galactosidase, beta-glucuronidase, or beta-lactamase. Alternatively, an enzyme can be used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Puhlication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. 0.1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will he appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR.

In some embodiments, the level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor tissue sample with a probe library, such that the presence of the target in the sample creates a probe pair—target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute level or normalized level. Typically, levels are normalized by correcting the absolute level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the level in one sample, e.g., a subject sample, to another sample, or between samples from different sources.

A further object of the present invention relates to a method for determining the survival time of a patient suffering a non small cell lung cancer (NSCLC) that coexists with (moderate to severe) chronic obstructive pulmonary disease (COPD) comprising
i) providing a tumor tissue sample from the patient,
ii) quantifying the density of CD8 T cells in the tumor tissue sample,
iii) comparing the density quantified at step ii) with a predetermined reference value
iv) determining whether the tumor tissue sample is positive or negative for PD-L1 expression and
v) concluding that the patient will have a long survival time when the density quantified at step ii) is higher than its corresponding predetermined reference value and the tumor tissue sample is negative for PD-L1 expression,
or concluding that the patient will have a short survival time when the density quantified at step ii) is lower than its predetermined value independently from the fact that the tumor sample is positive or negative for PD-L1 expression or the density quantified at step ii) is higher than its corresponding predetermined reference value and the tumor tissue sample is positive for PD-L1 expression.

The method is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

As used herein, the term “T cell” has its general meaning in the art and includes cells within the T cell lineage, including thymocytes, immature T cells, mature T cells and the like. Typically, T cells are characterized by expression of T cell markers at their cell surface. As used herein, the term “T cell marker” refers to surface molecules on the T cells which are specific for particular T cells. T cell markers suitable for use in the present invention include, but are not limited to surface CD3, CD4, CD8, CD45RO or any other CD antigen specific for T cells. As used herein the term “cytotoxic T cells” or “CD8 T cells” has its general meaning in the art and refers to a subset of T cells, once activated by a MHC-antigen complex, releases the protein perforin, which forms pores in the target cell's plasma membrane; this causes ions and water to flow into the target cell, making it expand and eventually lyse. Cytotoxic T cells also release granzyme, a serine protease that can enter target cells via the perforin-formed pore and induce apoptosis (cell death). Most cytotoxic T cells have present on the cell surface the protein CD8, which is attracted to portions of the Class I MHC molecule. In some embodiments, the quantification of density of CD8 T cells is determined by immunohistochemistry (IHC). For example, the quantification of the density of CD8 T cells is performed by contacting the tissue tumor tissue sample with a binding partner (e.g. an antibody) specific for a cell surface marker of said cells. Typically, the quantification of density of CD8 T cells is performed by contacting the tissue tumor tissue sample with a binding partner (e.g. an antibody) specific for CD8. Typically, the density of CD8 T cells is expressed as the number of these cells that are counted per one unit of surface area of tissue sample, e.g. as the number of cells that are counted per cm² or mm² of surface area of tumor tissue sample. In some embodiments, the density of cells may also be expressed as the number of cells per one volume unit of sample, e.g. as the number of cells per cm3 of tumor tissue sample. In some embodiments, the density of cells may also consist of the percentage of the specific cells per total cells (set at 100%).

Typically, it is considered that the tumor tissue sample is negative for PD-L1 expression when less than 1 percent of tumor cells express PD-L1. Conversely, it is considered that the tumor tissue sample is positive for PD-L1 expression when more than 1 percent of tumor cells express PD-L1.

In some embodiments, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of the gene in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the gene in a group of reference, one can use algorithmic analysis for the statistic treatment of the measured expression levels of the gene(s) in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of a) providing a collection of samples; b) providing, for each ample provided at step a), information relating to the actual clinical outcome for the corresponding subject (i.e. the duration of the survival); c) providing a serial of arbitrary quantification values; d) determining the expression level of the gene for each sample contained in the collection provided at step a); e) classifying said samples in two groups for one specific arbitrary quantification value provided at step c), respectively: (i) a first group comprising samples that exhibit a quantification value for level that is lower than the said arbitrary quantification value contained in the said serial of quantification values; (ii) a second group comprising samples that exhibit a quantification value for said level that is higher than the said arbitrary quantification value contained in the said serial of quantification values; whereby two groups of samples are obtained for the said specific quantification value, wherein the samples of each group are separately enumerated; f) calculating the statistical significance between (i) the quantification value obtained at step e) and (ii) the actual clinical outcome of the subjects from which samples contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant) has been calculated at step g).

For example the expression level of the gene has been assessed for 100 samples of 100 subjects. The 100 samples are ranked according to the expression level of the gene. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of the gene corresponding to the boundary between both subsets for which the p value is minimum is considered as the predetermined reference value.

It should be noted that the predetermined reference value is not necessarily the median value of expression levels of the gene. Thus in some embodiments, the predetermined reference value thus allows discrimination between a poor and a good prognosis for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level of the gene with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a subject may be assessed by comparing values obtained by measuring the expression level of the gene, where values higher than 5 reveal a poor prognosis and values less than 5 reveal a good prognosis. In some embodiments, a subject may be assessed by comparing values obtained by measuring the expression level of the gene and comparing the values on a scale, where values above the range of 4-6 indicate a poor prognosis and values below the range of 4-6 indicate a good prognosis, with values falling within the range of 4-6 indicating an intermediate occurrence (or prognosis).

A further object of the present invention relates to a method of treating a patient suffering from a non-small cell lung cancer (NSCLC) that coexists with chronic obstructive pulmonary disease (COPD) with a PD-1 inhibitor comprising i) determining the survival time by the method above mentioned and ii) administering to the patient a therapeutically effective amount of a PD-1 inhibitor when it is concluded at step i) that the patient will have a short survival time.

As used herein, the term “therapeutically effective amount” refers to an amount effective, at dosages and for periods of time necessary, to achieve a desired therapeutic result. A therapeutically effective amount of the immune checkpoint inhibitor of the present invention may vary according to factors such as the disease state, age, sex, and weight of the individual, and the ability of the immune checkpoint inhibitor of the present invention to elicit a desired response in the individual. A therapeutically effective amount is also one in which any toxic or detrimental effects of the antibody or antibody portion are outweighed by the therapeutically beneficial effects. The efficient dosages and dosage regimens for the immune checkpoint inhibitor of the present invention depend on the disease or condition to be treated and may be determined by the persons skilled in the art. A physician having ordinary skill in the art may readily determine and prescribe the effective amount of the pharmaceutical composition required. For example, the physician could start doses of the immune checkpoint inhibitor of the present invention employed in the pharmaceutical composition at levels lower than that required achieving the desired therapeutic effect and gradually increasing the dosage until the desired effect is achieved. In general, a suitable dose of a composition of the present invention will be that amount of the compound, which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of a inhibitor of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of a inhibitor of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labeled inhibitor of the present invention, fragment or mini-antibody derived from the inhibitor of the present invention. If desired, an effective daily dose of a pharmaceutical composition may be administered as two, three, four, five, six or more sub-doses administered separately at appropriate intervals throughout the day, optionally, in unit dosage forms. In some embodiments, the human monoclonal antibodies of the present invention are administered by slow continuous infusion over a long period, such as more than 24 hours, in order to minimize any unwanted side effects. An effective dose of a inhibitor of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of a inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the immune checkpoint inhibitor of the present invention is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a patient, the composition will be formulated for administration to the patient. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1: Immune cell prognostic value according to patients' COPD status and to PD-L1 expression by tumor cells. (A) and (B) Kaplan-Meier curves of OS in NSCLC patients according to PD-L1 expression by tumor cells, in non-COPD (n=234) and in COPD⁺ patients (n=192), respectively. (C) Kaplan-Meier curves of OS in NSCLC patients according to CD8Tu cell density (median CD8Tu cell density) and PD-L1 expression by tumor cells. (D) and (E) Kaplan-Meier curves of OS in the CD8Tu^{High} group of patients, without COPD (n=123) and with COPD (n=91), respectively, according to PD-L1 expression by tumor cells. (F), (G) and (H) forest plots of univariate Cox-regression analyses showing the impact of CD8Tu cell, CD8s cell and DC-Lamp′ cell density on the OS according to PD-L1 expression by tumor cells, in the whole cohort, in non-COPD and in COPD⁺ patients, respectively.

FIG. 2: Anti-PD-L1 treatment enhances lymphocyte infiltration and reduces LLC lung tumor burden in smoke exposed mice. A) Schematic representation of the experimental design. Six- to 8-week-old C57/B16 were exposed to cigarette smoke for 8 weeks and then inoculated with 10⁵ LLC cells via tail vein injection. Anti-PDL1 antibody and Rat IgG2b isotype control were i.p injected from day 5 after tumor challenge every 3 days (d5, d8, d11 and d14) at 200 μg/mouse. On day 17 after tumor challenge, lungs were harvested and embedded with Bouins fixative to count surface nodules (group 1). From each cohort the lungs from 5 mice were used for gene expression and for TIL analysis by flow cytometry (group 2). B) Anti-PD-L1, but not isotype control antibody, reduced lung tumor burden. The anti-tumor effect of PD-L1 blockade was more prominent in mice that were exposed to chronic cigarette smoke (RA-LLC anti-PD-L1 versus SM-LLC anti-PD-L1). Data are represented as mean+/−SD, n=6-13 mice per group. C) and D) Lungs were harvested and digest for FACS analysis (from group 2). Lymphocyte staining was performed with surface markers CD45, CD8, CD4, CD3, CD19 and NK1.1. Dead cells were excluded and cells were gated on CD45 positive cells and SSC^{low} in order to determine the lymphocytes gate. C) Number of total lung lymphocytes, CD8 T cells, CD4 T cells, B cells and NK cells. D) Percentage of lung lymphocyte subtypes of total lymphocytes. Anti-PD-L1 treatment altered the distribution of lung lymphocyte subsets in smoke exposed mice, but not in room air exposed mice, and increased the number of lung T and NK cells in tumor bearing mice exposed to cigarette smoke, relative to control treated animals. Data are represented as mean+/−SD, n=5 mice per group for lymphocyte percentages and mean+/−SEM for lymphocyte numbers.

### EXAMPLE: 1: MATERIAL & METHODS

Patients

Our retrospective cohort includes 435 patients with NSCLC, seen between June 2001 and December of 2005 at the department of Thoracic Surgery of Hotel-Dieu hospital (Paris, France). Patients underwent a complete surgical resection of their lung tumor and Formalin-Fixed Paraffin-Embedded (FFPE) primary lung tumor samples were obtained for each of them. Patients with previous lung cancer or other synchronous cancers, and patients treated by neoadjuvant chemotherapy or radiotherapy were ineligible. At the completion of this work, the minimal follow-up was 80 months for the last patient included in the cohort. The observation time of the cohort was the interval between tumor resection and the last contact (last follow-up or death of the patient). Data on long-term survival were obtained retrospectively by interrogation of municipal registers. Fresh tumor biopsies, distant non tumoral lung samples, and peripheral blood were retrieved from 51 patients with untreated NSCLC who underwent surgery between March 2014 and December 2015. These samples were used to perform flow cytometry experiments. Written informed consent before inclusion in the study was obtained for all patients. The protocol was approved by the local ethics committee (CPP Ile de France II, n° 2008-133 and 2012 06-12) in application with article L.1121-1 of French law.

COPD Assessment

The Global Initiative for Chronic Obstructive Lung Disease (GOLD 2007) criteria were used to assess the presence of COPD and to evaluate disease severity (64). Since most of our patients were operated between June 2001 and December 2005, the new stratification (GOLD 2011) including also the level of daily symptoms, in particular dyspnea and the history of exacerbations was inapplicable. Spirometry was used to determine the Forced Expiratory Volume in 1 second (FEVi) and the Forced vital capacity (FVC). Only subjects with a post-bronchodilator FEVi/FVC ratio of less than 0.70 were considered as having a COPD. The severity of airflow obstruction was assessed by evaluating the FEVi expressed as a percentage of a normal predicted value (FEVi % predicted). Accordingly, among the 435 patients of the retrospective study, 197 patients had a COPD, of whom 57 patients had mild COPD⁺ GOLD stage I (FEV₁>80% predicted), 119 patients had moderate COPD GOLD stage II (50%≤FEV₁%<80) and 21 patients had severe COPD GOLD stage III (30≤FEV₁%<50).

Immunohistochemistry

For each FFPE lung tumor sample, two observers, including at least one expert pathologist, selected the tumor section containing the highest density of immune cells on hematoxylin and eosin-safran-stained slides. Serial 5 μm tissue sections were deparaffinized, rehydrated and pretreated in appropriate buffer for antigen retrieval. Slides were then incubated with 5% human serum (ref. S4190, Biowest) for 30 min at room temperature. Tissue sections were incubated for one hour at room temperature with the following primary antibodies polyclonal anti-CD3 (Dako), anti-CD8 (SP16, Spring-bioscience), anti-DC-Lamp (1010.01, Dendritics), anti-CD66b (G10F5, BD bioscience) or anti-CD68 (PG-M1, Dako), followed by an incubation with the appropriate biotinylated secondary antibodies for 30 minutes at room temperature, and with peroxidase-conjugated streptavidin (Dako) for 30 min at room temperature. For PD-L1 staining, anti-PD-L1 (E1L3N, Cell signaling) antibody was incubated for 3 hours at room temperature and then incubated for 30 minutes with SignalStain® Boost IHC Detection Reagent (HRP, Rabbit, Cell signaling). The enzymatic activity was revealed as previously described (65). For single stainings, sections were counterstained with hematoxylin. Slides were scanned using a Nanozoomer (Hamamatsu) operated with NDPview software.

Method for Cell Quantification

Using Calopix software (Tribvn), CD66b⁺ and CD68⁺ cells were counted on the whole tumor section, while CD8⁺ cells were counted separately in the tumor nests and in the tumor stroma. DC-Lamp⁺ cells were counted manually in the whole tumor section. Areas of the whole tumor section, of tumor nests and of tumor stroma were determined using Calopix software. For CD66b⁺, CD68⁺, CD8⁺ and DC-Lamp⁺ cells results were expressed as an absolute number of positive cells/mm². The percentage of PD-L1⁺ cells among tumor cells was determined manually. The positivity threshold was fixed at ≥1%. Both immunostaining and quantification were reviewed by at least two independent observers (JB, HO, or DD).

Cell and Tissue Preparation

Surgical samples, including fresh lung tumor biopsies and distant non-tumoral lung samples were mechanically dilacerated. A non-enzymatic disruption in the Cell Recovery Solution (Corning) for 1 hour at 4° C. was performed. Cells were then filtered through a 70 μm cell strainer (BD Biosciences). Mononuclear cells from peripheral blood, distant non-tumoral lung and tumor tissue were isolated using a Ficoll gradient.

Flow Cytometry

Cells were incubated for 30 min at 4° C. in PBS 0.5 mM of EDTA containing 2% of human serum to block the Fcγ receptors. Then, surface cells were stained with appropriate dilutions of various monoclonal antibodies or the appropriate isotype controls, for 30 minutes at 4° C. in the dark. Cells were washed and fixed in PBS 0.5% formaldehyde before the analysis.

For intracellular cytokine staining, cells were stimulated for 4 h with phorbol 12-myristate 13-acetate (PMA) and ionomycin (Sigma-Aldrich) in presence of Brefeldin A and Monensin (BD Pharmingen). In negative control tubes, Brefeldin A and Monensin were not added. Cell surface staining was performed with appropriate dilutions of various monoclonal antibodies or the appropriate isotype controls for 30 minutes at 4° C. in the dark. Cells were then permeabilized using the Fixation/Permeabilization Solution (BD Biosciences) and stained with appropriate dilutions of various monoclonal antibodies for 30 min at 4° C. in the dark. Flow cytometry acquisition was performed on a fifteen-color Fortessa cytometer (Becton Dickinson). Dead cells were excluded based on forward and side scatter characteristics. Reported statistical data are based on at least 500 events gated on the population of interest. Results were analyzed using DIVA (Becton Dickinson) and/or Flow Jo software (TreeStar, Inc). Flow Jo was used for graphical representations.

Statistical Analysis

Descriptive statistics involved the use of frequencies for qualitative variables and of mean (SD) or median (Q1-Q3) as appropriate for quantitative variables. Because of their distribution, immune cell densities were log-transformed. Categorical data were compared using a Chi-square test as soon as possible. In the opposite case a Fisher exact test was used. Categorical data were compared according to COPD stages using exact Cochran-Armitage trend test. According to data distribution, a parametric test (ANOVA, student's t test) or a non-parametric test (Kruskal-Wallis, Mann-Whitney), with appropriate post-hoc comparisons, was used to compare quantitative variables across the different groups. Correlations between quantitative parameters were performed using the Spearman test and correlation matrixes were represented with Genesis software (Institute of Genomics and Bioinformatics, Gratz, Austria). Survival analyses were performed using both log-rank test and Cox proportional-hazard regression model. The start of follow-up for OS was the time of surgery. Patients were followed up to death or to August 2013. All patients alive at this date were censored. When a log-rank test was performed, prognostic value of continuous variables was assessed using a median-based cut-off or quartile stratification. The median density was of 119 cells/mm² for CD8Tu cells and of 1.95 cells/mm² for DC-Lamp⁺ cells (Figure S2K). For Cox proportional-hazard models, immune cell densities were log-transformed. Parameters significantly different according to the COPD status or identified in univariate analysis as possibly influencing the OS (p<0.05) were introduced in a multivariate Cox-proportional hazard regression model. In multivariate analysis, variables intrinsically correlated (for instance, CD8Tu cell, CD8s cell and DC-Lamp⁺ cell densities) were not included in the same model. Multivariate analysis for OS was adjusted for age, gender, vascular emboli, smoking history and stratified on the stage of the tumor. Hypothesis of proportional hazards was systematically checked. Analyses were performed with Prism 5 (Graphpad), Statview software (Abacus Systems) and R (http://www.r-project.org/).

Cigarette Smoke and Lung Cancer Model

The murine Lewis Lung carcinoma (LLC) cell line (American Type Culture Collection) was propagated in Dulbecco's Modified Eagle Medium (DMEM) supplemented with 10% FBS, 1 mmol/L sodium pyruvate, 2 mmol/L L-glutamine, 10 μg/mL penicillin/streptomycin, and 0.1 mmol/L nonessential amino acids (Life Technologies). The LLC cell line was tested and validated to be mycoplasma free. Before i.v. injection, LLC cells were washed in sterile PBS and tested for viability. Several pilot experiments were conducted to test different numbers of LLC cells injected (from 10⁵ to 10⁶ cells given via tail vain injection) and duration of in vivo tumor growth in C57BL/6 mice to establish optimal conditions for the lung cancer model. Based on the results from these studies subsequent experiments were conducted with 10⁵ cells injected and a duration of in vivo lung tumor growth of 17 days. To test the effects of chronic smoke induced lung inflammation on tumor growth, 6-12 mice/group were exposed to the mainstream cigarette smoke (Reference Cigarette 3R4F without filter; University of Kentucky, Lexington, Ky., USA) of 18 cigarettes 2 times a day with 2 hour smoke-free intervals, 5 days a week for eight weeks using a whole body smoke exposure system (SIU-48, Promech lab AB). The control groups were exposed to room air for eight weeks. Groups of naïve mice and mice exposed to cigarette smoke but without LLC tumor challenge were included to control for the effects of smoke exposure. After two months of chronic smoke exposure 10⁵ LLC cells were injected intravenously (via tail-vein) into 6- to 8-week-old female C57BL/6 mice (Harlan). 17 Days later, lungs were used for counting of the number of tumor nodules using a dissecting microscope and to generate cell suspensions for FACS staining. To test the anti-tumor effect of anti-PD-L1 antibody treatment, mice were exposed to cigarette smoke for eight weeks and challenged with LLC as described above. Starting from 5 days after tumor cell inoculation, PD-L1 blockade was carried out using the anti-mouse PD-L1 antibody clone 10F (Bioxcell) or rat IgG2b isotype control (Bioxcell). Antibodies were administrated by intraperitoneal (i.p.) injection twice a week for two weeks at 200 μg/dose.

Characterization of Immune Cells by FACS Analysis

Intra-orbital puncture was used to collect blood and serum. Lungs from tumor bearing mice were perfused with PBS to removed blood and lavaged in situ to remove alveolar macrophages and collect BAL fluid. Infiltrating leukocytes were isolated from minced tumor and lung tissues from LLC-challenged mice by incubation in DMEM containing collagenase-III (2 mg/mL; Roche) and DNase I (1 mg/mL; Sigma Chemical) at 37° C. for 30 minutes. The Enzyme digestion was stopped by adding DMEM containing 10% FBS and the cell suspension was filtered through a 70-μm cell strainer. Cells were blocked with anti-FcR (clone 2.4G2, BD pharmingen) and stained with antibodies against PD-L1, PD-1, CD45, CD11b, Ly6C, Ly6G, for MDSCs, CD11c, MHCII, CD68, F4/80, CD40, CD86, CD80, CD206 for macrophages and dendritic cells (DCs), CD8, CD4, CD3e, NK1.1, CD69, CD44, CD62L, CD19, Tim3, ICOS, GL7, CD25 for lymphocytes. Dead cells were stained with a dead cell marker (blue dead, life tech) and excluded from the analysis. Samples were collected on a FACSCalibur Flow Cytometer (Becton Dickinson) and data were analyzed using Flow Jo software (Tree Star Inc.).

Proliferation and Cytokines Measurement:

KI-67, iNOS, and IFN-γ were evaluated by intracellular staining after fixation and permeabilization (BD cytofix as per manufacturer instructions). FoxP3 expression was quantified after permeabilization using the FoxP3 kit (ebioscience).

Histology and Immunohistochemical Detection of PD-L1 and F4/80 Expression.

Mice were euthanized and lungs were inflated with 10% of formalin and embedded in paraffin section. Sections (5 μm) were cut and stained for hematoxylin and eosin (H&E). We detected lung PD-L1 expression on paraffin sections using a polyclonal rabbit antibody to mouse PD-L1 antibody (Novus biological) overnight in a humidifier chamber in 1% goat serum and BSA to decrease non specific binding. A secondary goat anti-rabbit IgG (Vectastain ABC-alkaline phosphatase kit system, Vector laboratories) was used together with the fast red substrate (Vector laboratories) for detection. We detected lung F4/80 expression on paraffin section by using a monoclonal rat antibody to F4/80 (Abcam, 1 ug/ml final) in 1% rabbit serum and BSA to decrease non specific binding. A secondary biotinynaled rabbit anti-rat IgG antibody and the Vector black/brown substrate were used for detection. To test for the specificity of immunostainings, primary antibodies were replaced with rabbit normal isotype (Vector) for PD-L1 staining and a rat isotype control for F4/80 staining at the same dilution.

RNA Extraction and Fluidigm RT-PCR Analysis Assay

RNA was extracted with the miRNeasy kit (Qiagen). RNA quantity and quality were assessed using Nanodrop (Thermo Scientific, Waltham, Mass.) by the ratio D0260/280 and by Agilent 2100 Bioanalyzer (Agilent Technologies, Santa Clara, Calif.) by determining RIN score and the rRNA ratio 28S/18S. RNA from lung samples was reverse transcribed into cDNA using SuperScript III First-Strand Synthesis SuperMix kit (Life Technologies, Carlsbad, Calif.). The reaction contains mRNA (800 ng in 6 μL), 1 μl of random hexamers and 1 μl of annealing buffer to each RNA sample. The reverse transcription was performed at 65° C. for 5 min, and a second mix composed of 10 μl of 2× First Strand Buffer and 2 μl of SuperScript III was added (20 uL total reaction volume). The reaction was performed at 25° C. for 10 min, 50° C. for 15 sec and 85° C. for 5 min. The cDNA was stored at −20 C. For pre-amplification of cDNA, we pooled 91- or 96 TaqMan Assays at a final concentration of 0.2× for each assay. Pre-PCR amplification reaction was done at 10 μl containing 5 μl TaqMan PreAmp Master Mix (2×), 2.5 μl of 91- or 96-pooled TaqMan assay mix (0.2×) and 2.5 μl of cDNA. The pre-amplification PCR performed at one cycle 95° C. for 10 min, 10 cycles at 95° C. for 15 sec and then 60° C. for 4 min. After pre-amplification PCR, the product was diluted 1:10 with TE buffer and stored at −80° C. until needed.

Real-Time qPCR

Quantitative RT-PCR was carried out using Real-time PCR system in a 96 wells plate format. Before putting assays and samples on the chip, control line fluid was injected into each accumulator of the chip. Then, the chip was placed into the IFC (integrated fluidic circuit) controller, then run the Prime (136×) script to prime the control line fluid into the chip (40 min). When the Prime (136×) script has finished, the primed chip was removed from the IFC controller and pipette 5 μL of PCR reaction of each assay and each sample into their respective inlets on the chip. For assays, PCR reaction of 5 μl contained 2.5 μl of 91 pooled TaqMan Assays at a final concentration of 0.2× and 2.5 μl of 2× Assay Loading Reagent (Fluidigm, PN 85000736). For samples, the PCR reaction of 5 μl contained 2.5 μl of TaqMan PCR Master Mix-(2×) (Applied Biosystems, PN 4304437), 0.25 μl GE sample loading reagent (20×) (Fluidigm, PN 85000746), 2.25 μl of diluted preamplified cDNA. Samples were run and analyzed on BioMark 96.96 Dynamic Array chips with the BioMark Real-Time PCR System. The PCR was performed at 95° C. for 10 min, followed by 40 cycles at 95° C. for 15 sec and 60° C. for 1 min. Three technical replicates were run for each sample in a 96 format plate and a total of 3 plates run in this study. On each plate, three endogenous control genes (HPRT, GAPDH, and UBC) were included.

Statistical Analysis for Lung Cancer Model Studies

Data were analyzed using Prism 6.0 software (Graph Pad Software). Data are represented as the mean+/−SEM or SD (see figures) using nonparametric tests (Kruskall-Wallis, Mann-Whitney U. P values less than 0.05 were considered to be significant.

### EXAMPLE 2: RESULTS IN NSCLC PATIENTS

Similar Immune Cell Densities in NSCLC Patients with and without COPD.

Tumors samples from a retrospective cohort of 435 patients with NSCLC, who did not receive neo-adjuvant chemotherapy, were used to determine by quantitative immunohistochemistry the densities of the following immune cell populations: neutrophils (CD66b⁺ cells), macrophages (CD68⁺ cells), mature DCs (DC-Lamp⁺ cells), and CD8 T cells in the tumor nests (CD8Tu) and in the stroma (CD8s). Among the 435 patients, 197 had COPD, including 57 patients with COPD GOLD stage I (COPD⁺ I), 119 patients with COPD GOLD stage II (COPD⁺ II) and 21 patients with COPD GOLD stage III (COPD⁺ III). As expected, the mean age of patients, the frequencies of male patients and that of smokers were higher within the COPD⁺ group as compared to non-COPD patients. Interestingly, overall patients' survival was not significantly different according to the COPD status. However, we confirmed that coexisting COPD was associated with worse survival in NSCLC stage I patients (39). Of note, due to the small number of patients in the COPD⁺ III group, the COPD⁺ II and COPD⁺ III groups were merged together for most of the subsequent analyses. Finally, no significant differences could be observed between COPD⁺ and non-COPD patients regarding the mean densities of the four tumor-infiltrating immune cell populations studied, regardless of the COPD GOLD status.

Absence of Immune Cell Prognostic Value in NSCLC Patients with Coexisting COPD.

To investigate indirectly the functionality of immune cells in the tumor microenvironment, we determined their impact on patient survival. Since a high density of CD8_{Tu} cells is clearly correlated with long-term survival in NSCLC (14, 41), we first studied their prognostic value according to the COPD status of the patients. We segregated patients from our retrospective cohort into two groups according to the median density of CD8Tu cells. In non-COPD patients, a high CD8Tu cell density was associated with a longer overall survival (OS). The median OS was 62 months for the CD8Tu^{low} group and increased up to 95 months for the CD8Tu^{high} group. However, a high density of CD8Tu cells was not significantly associated with a better survival in the COPD⁺ group. Remarkably, this result was even more pronounced in COPD⁺ II-III patients with a difference in terms of median OS of only six months between the CD8Tu^{low} group (63 months) and the CD8Tu^{high} group (69 months). Then by using the quartile stratification, we observed in non-COPD patients that CD8Tu cell density was associated with a longer OS as soon as the 2^{nd} quartile was reached, while in COPD⁺ II-III patients the survival curves for all quartiles merged together. In parallel, we also studied the prognostic impact of DC-Lamp⁺ cells, which reflect the presence of TLS, according to the COPD status of the patients. In contrast to non-COPD patients or COPD⁺ I patients, DC-Lamp⁺ cell density was not associated with a significant prognostic value in COPD⁺ II-III patients. Next, we performed univariate Cox-regression analysis to decipher, according to the COPD status of the patients, the prognostic value of the four tumor-infiltrating immune cell populations studied. While a non-significant trend toward a favorable prognostic value of a high density of neutrophils was observed only in non-COPD patients, macrophage density was not associated with any prognostic value, whatever the group considered. In contrast, CD8Tu cell, CD8s cell and DC-Lamp⁺ cell densities were all associated with a good prognostic value in non-COPD patients, while in COPD⁺ patients only DC-Lamp⁺ cell density was associated with improved survival. However, the prognostic value of DC-Lamp⁺ cell density was lost in COPD⁺ II-III patients. All of these results were confirmed by multivariate Cox-regression analysis adjusted for age, gender, vascular emboli, smoking history and stratified on tumor stage.

The Frequency of PD-1⁺ TIM-3⁺ Cells Among CD8 TILs Reflects Exhaustion Severity and is Higher in COPD II-III Patients.

Based on above results, we investigated whether effector functions of CD8 TILs were altered in COPD patients. We first conducted a large-scale flow cytometry analysis to characterize CD8 TILs in the context of NSCLC by following a gating strategy. Within the tumor tissue (Tumor), we observed a predominance of effector memory CD8 T cells (T_{EM} cells) and a lower percentage of terminally differentiated effector memory CD8 T cells (TEMRA cells) among total CD8 T cells, relative to other anatomical sites, such as blood and non-tumor distal lung tissue (NT). Naïve CD8 T cells (T_{N} cells) were virtually absent in Tumor and the proportion of central memory CD8 T cells (T_{CM} cells) was higher in blood, while no significant difference was observed between Tumor and NT. Regarding cytokine secretion, the frequencies of CD8 T cells able to secrete Granzyme B, TNF-α, IFN-γ and IL-17 were significantly lower in Tumor compared to NT. Then, we studied the expression of the four following immune checkpoints: CTLA-4, LAG-3, PD-1 and TIM-3. Although CTLA-4 was weakly expressed, the percentage of CTLA-4⁺ cells among CD8 T cells was higher in Tumor than in blood. Strikingly, higher proportions of CD8 T cells expressing LAG-3, PD-1 and TIM-3 were found within the tumor tissue. TIM-3 expression in particular appeared to be nearly restricted to CD8 TILs, while LAG-3 and PD-1 were found to be also expressed by CD8 T cells in NT, and to a lesser extent in blood. TIM-3 and PD-1 were mainly expressed by T_{EM} cells and T_{CM} cells. In the Tumor, PD-1 and TIM-3 expression by CD8 TILs were strongly positively correlated, as well as frequencies of IFN-γ⁺ and TNF-α⁺ cells. Remarkably, CD8 TILs co-expressing PD-1 and TIM-3 were restricted to Tumor, and their frequency among CD8 TILs was inversely correlated with those of IFN-γ⁺ and TNF-α⁺ cells. The percentage of cells, among CD8 TILs, expressing TIM-3 but not PD-1 was still negatively associated with the frequency of cells capable of secreting TNF-α, whereas this association was not observed with PD-1⁺ TIM-3⁻ cells. In accordance with this observation, the concomitant analysis of exhaustion marker expression and cytokine production in the tumors of 10 NSCLC patients revealed that CD8 TILs expressing both PD-1 and TIM-3 were those with the lowest secretion of TNF-α and IFN-γ. No association between the frequency of Granzyme B⁺ cells and PD-1/TIM-3 expression could be observed. The co-expression of PD-1 and TIM-3 thus reflects CD8 TIL exhaustion in NSCLC. We then evaluated the link between the frequency of PD-1⁺ TIM-3⁺ cells among CD8 TILs and COPD severity. Airflow obstruction severity is inversely correlated with the FEV1 expressed as a percentage of a normal predicted value (FEV1% predicted) (see materials and methods section). Here we observed that in COPD patients the FEV1% predicted was inversely and significantly correlated with the proportion of CD8 TILs co-expressing PD-1 and TIM-3, meaning that COPD severity was positively associated with CD8 TIL exhaustion. In addition, a higher frequency of TIM-3⁺ cells among CD8 TILs was observed in COPD⁺ II-III patients as compared to non-COPD patients. Moreover, the frequency of PD-1⁺ and of TIM-3⁺ cells among T_{CM} cells was higher in COPD⁺ II-III patients as compared to the other groups. Finally, a higher percentage of cells co-expressing PD-1 and TIM-3 among CD8 TILs and among T_{EM} cells was also observed in COPD⁺ II-III patients.

Higher CD8 TIL Exhaustion in Heavily Infiltrated Tumors is Dramatically Exacerbated in COPD⁺ Patients.

We then investigated the relationship between CD8 TIL exhaustion and their density in the tumor. Consequently, we segregated patients into two groups according to CD8Tu cell median density determined by performing immunohistochemistry on the corresponding FFPE slides. On the whole prospective cohort, the frequencies of CD8 TILs expressing PD-1, TIM-3 and co-expressing PD-1/TIM-3 were dramatically higher in CD8Tu^{high} patients. On the opposite, the proportion of CD8 TILs secreting TNF-α and IFN-γ was lower in this group. With regard to TIM-3 expression and cytokine secretion, similar data were obtained when patients were stratified according to the CD8s cell density, while no difference could be observed when patients were stratified according to DC-Lamp⁺ cell density. Since CD8Tu cell density was the one most related to exhaustion, we studied the correlation between this parameter and the level of CD8 TIL exhaustion according to patients' COPD status. CD8Tu cell density and CD8 TIL exhaustion, including the frequencies of CD8 TILs expressing PD-1, TIM-3 and co-expressing PD-1/TIM-3, were more strongly correlated in COPD⁺ patients as compared to non-COPD patients. Accordingly, the frequency of TNF-α⁺ cells among CD8 TILs was significantly and negatively associated with CD8Tu cell density only in COPD⁺ patients. Interestingly, in COPD⁺ patients and as compared to non-COPD patients, CD8 TIL exhaustion increased as soon as an intermediary CD8Tu cell density was reached (250-400 cells/mm²). Obviously, the strong impact of immunosuppression on tumor burden is based on TILs exhaustion, but also on concomitant mechanisms developed by malignant cells to avoid immune surveillance, for instance by expressing ligands to the so-called immune checkpoints. We then investigated whether the orchestration of immunomodulation in the tumor microenvironment differs in COPD patients. More precisely, we studied the relationship between immune cell densities, CD8 TIL exhaustion and PD-L1 expression by malignant cells in the tumor according to the COPD status of the patients. To do so, we evaluated by immunohistochemistry in the aforementioned prospective cohort the expression of PD-L1 by tumor cells, but also the densities of macrophages and neutrophils, on the corresponding FFPE slides. The percentage of tumor cells expressing PD-L1 was determined as described in the material and methods section. Flow cytometry data characterizing CD8 TILs, immune cell densities and the percentage of tumor cells expressing PD-L1 were all included in the correlation matrixes. In the non-COPD group a strong association between the densities of macrophages, CD8Tu cells, CD8s cells, DC-Lamp⁺ cells and the frequency of tumor cells expressing PD-L1 was observed (cluster-1a). This cluster was then associated with the expression of PD-1 and TIM-3 by CD8 TILs (cluster-2a). In the COPD⁺ group, CD8Tu cell and CD8s cell densities were directly correlated with the frequency of cells expressing PD-1 and TIM-3 among CD8 TILs, and with PD-L1 expression by tumor cells (cluster-1b). In contrast to the non-COPD group, DC-Lamp⁺ cell density did not cluster directly with CD8 T cell densities in NSCLC samples from patients with COPD. We then segregated the non-COPD patients and the COPD⁺ patients into CD8Tu^{high} and CD8Tu^{low} groups according to the whole prospective cohort CD8Tu cell median density. In both subgroups the level of PD-L1 expression by tumor cells was higher among CD8Tu^{high} patients. In COPD⁺ patients, the frequencies of cells expressing PD-1, TIM-3, and co-expressing PD-1 and TIM-3 among CD8 TILs were higher in the CD8Tu^{high} group as compared to the CD8Tu^{low} group, while the proportion of CD8 TILs secreting TNF-α was decreased. In the non-COPD group, these differences were less significant and limited to the frequencies of TIM-3⁺ cells and of PD-1⁺ TIM-3⁺ cells among CD8 TILs. Finally, in the CD8Tu^{high} group, the frequency of CD8 TILs co-expressing PD-1 and TIM-3 was higher in COPD⁺ patients as compared to non-COPD patients, while an opposite but non-significant trend was observed for TNF-α secretion (p=0.09).

PD-L1 Expression by Tumor Cells is Associated with a Shorter Survival Mainly in COPD⁺ Patients with an Active Tumor Immune Microenvironment.

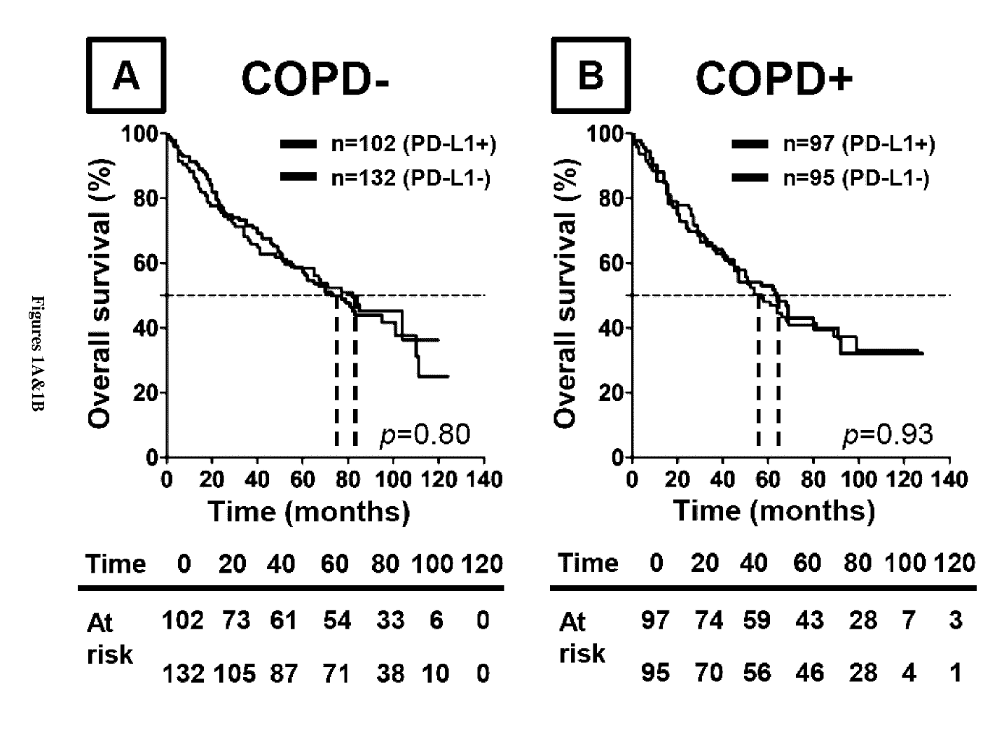

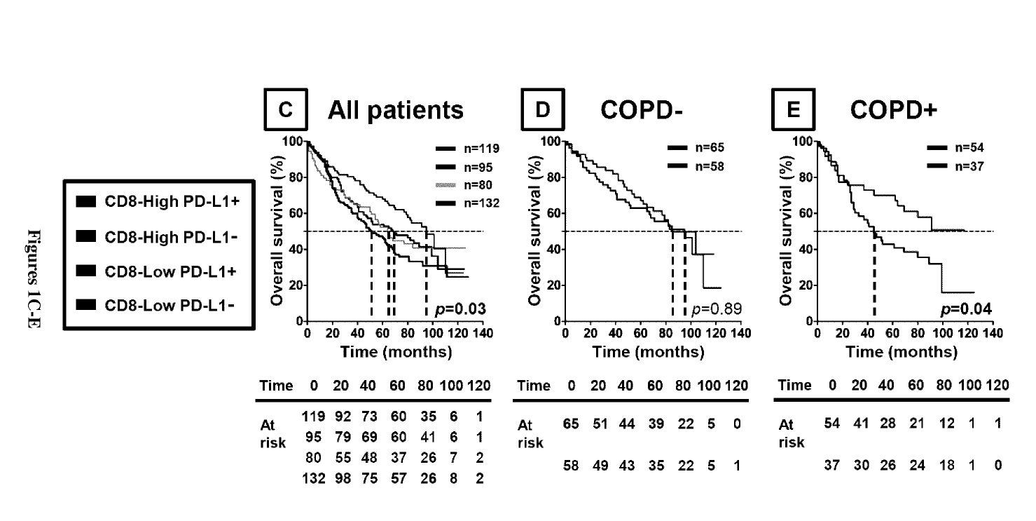

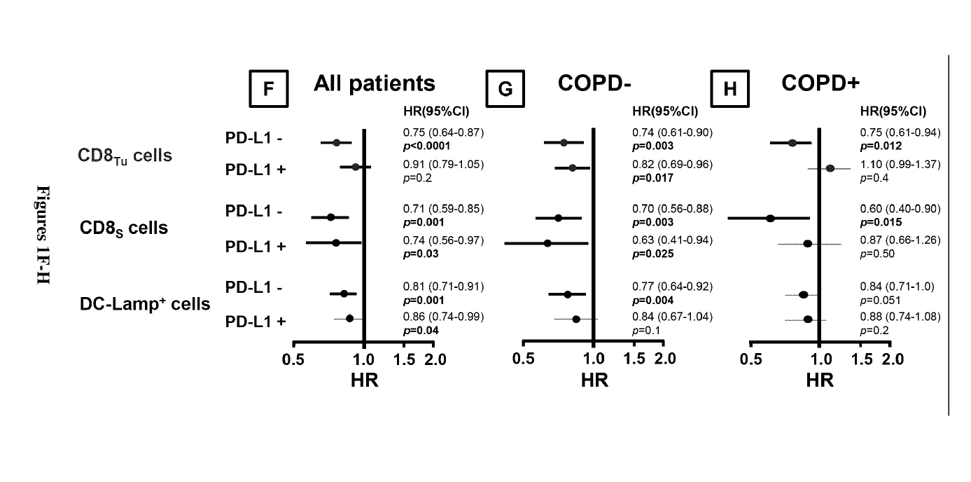

We investigated the impact of PD-L1 expression by tumor cells on the OS of NSCLC patients according to their COPD status. No significant difference of expression could be observed according to the COPD status of the patients. Whatever the group of patients considered, PD-L1 expression by tumor cells was not associated with a significant prognostic value (FIGS. 1A and B). Further, we confirmed in the whole retrospective cohort the results previously observed, meaning that the frequencies of tumor cells expressing PD-L1 was dramatically higher in the CD8Tu^{high} group, the CD8s^{high} and the DC-Lamp^{high} groups. Consequently, we then determined whether PD-L1 prognostic value may depend on the CD8Tu cell density. As shown in FIG. 1C, PD-L1 expression was associated with a bad prognosis mostly in the CD8Tu^{high} group, with a median OS of 95 months for CD8Tu^{high} PD-L1⁻ patients, while the median OS of CD8Tu^{high} PD-L1⁺ patients was of 68 months. Interestingly, this association was even more pronounced in COPD⁺ patients (FIG. 1E), but was not confirmed in non-COPD patients (FIG. 1D). We then performed univariate Cox-regression analysis to study CD8Tu cell, CD8s cell and DC-Lamp⁺ cell prognostic value according to PD-L1 expression by tumor cells (FIGS. 1F, 1G and 1H). When considering the whole retrospective cohort, PD-L1 expression negatively impacted the prognostic value of CD8Tu cells, CD8s cells and DC-Lamp⁺ cells (FIG. 1F). In non-COPD patients, the prognostic values of CD8Tu cell and of CD8s cell densities were similar whatever PD-L1 expression by tumor cells (FIG. 1G). Remarkably, in COPD patients, CD8Tu cell (HR=0.75; p=0.012) and CD8s cell (HR=0.60; p=0.015) densities were positively associated with a better OS in tumors which did not express PD-L1, while these prognostic values were totally lost in the PD-L1⁺ groups, with a HR of 1.1 for CD8Tu cell (p=0.4) and of 0.87 for CD8s cell (p=0.5) (FIG. 1H). Finally, when subgroups of patients were defined according to a cut-off of PD-L1⁺ tumor cell frequency ≥5% and ≥10%, the prognostic values of CD8Tu cell and of CD8s cell densities were similarly lost in patients with the highest expression of PD-L1. Remarkably, these results were even more pronounced in COPD⁺ patients and not observed in non-COPD patients.

### EXAMPLE 3: RESULTS ON ANIMAL MODELS

Smoke Exposure Increases Tumor Burden and Changes Lymphocyte Infiltration and Activation in the Lewis Lung Carcinoma (LLC) Lung Tumor Model

Exposure to cigarette smoke is a major risk factor for both COPD and lung cancer and patients with COPD have a significantly increased risk of developing NSCLC. While noxious agents contained in cigarette smoke may lead to oncogenic mutations, it is also recognized that chronic inflammatory conditions can facilitate or promote tumor growth. However, the key components of smoke exposure or COPD related inflammation that promote tumor growth are still poorly understood. Also the effects of chronic lung inflammation on the outcome of treatment with checkpoint inhibitors, new therapeutic modalities that have shown promising results in lung cancer and other indications, has not yet been studied in detail. Here we studied the impact of chronic cigarette smoke exposure on inflammatory parameters in the lung as well as on Lewis Lung Carcimoma (LLC) tumor burden. To avoid any direct impact of smoke exposure on LLC tumor cells, C57BL mice were first subjected to 8 weeks of chronic smoke exposure. The day following smoke cessation, LLC cells were injected via the tail vain and 17 days later tumor burden was assessed by counting the number of tumor nodules. Lungs were treated with Bouins fixative to better visualize nodules for quantification. In repeated experiments, precondition with 8 weeks of smoke exposure resulted in significantly increased tumor burden. CD45-negative cells from the lungs of smoke exposed mice also showed an increase in the percentage of Ki67+ cells, indicating an enhanced proliferative capacity of LLC cells in these animals. The results demonstrate that preconditioning with chronic cigarette smoke enhances LLC tumor growth and increases overall tumor burden. Cigarette smoke alone did not significantly change the relative frequency of lymphocytes in lung tissue, as assessed by flow cytometry. The combination of smoke exposure with LLC tumor challenge did, however, result in an increase in the percentage of CD8 T cells among lymphocytes. Tumor challenge also increased the percentage of CD4 T cells and NK cells, an effect that was reduced in smoke exposed mice. Further analysis revealed an altered activation status of lung immune cells in smoke exposed mice. In particular for B cells and CD8 T cells the percentages that showed up-regulation of activation markers were increased. In some instances the effect was further enhanced in tumor bearing mice (for example CD69 expression on CD19+ B cells and CD40 ligand expression by CD8 T cells). While tumor challenge increased the frequency of CD69+ NK cells this was not seen in tumor bearing mice exposed to cigarette smoke. Smoke exposure resulted in a modest, but statistically significant, increase in lung dendritic cells (DCs). This effect was more pronounced in tumor bearing animals that had been subjected to smoke exposure. DCs from tumor bearing mice also showed enhanced expression of activation and maturation markers CD40, CD80 and CD86. Interestingly, expression of CD80 and CD86 was reduced in smoke exposed mice as compared to controls. Together, the data show that smoke exposure, and even more so the combination of smoke with tumor challenge, results in a more activated immune environment in the lung, in particular with regard to T cell activation.

Smoke Exposure Modulates the Expression of the PD-1/PDL-1 T Cell Checkpoint in COPD Lung Tumor Model

The flow cytometric analysis revealed an increase in the frequency of CD8 T cells expressing activation markers in LLC tumor bearing mice that were subjected to chronic smoke exposure. While CD8 T cells are thought to be critical for an anti-tumor immune response (Pardoll, 2012), the tumor burden in smoke exposed mice is greater than in sham treated mice. T cell responses are regulated by both co-stimulatory (such as ICOS) pathways as well as co-inhibitory mechanism. One co-inhibitory pathway that has gained significant attention recently is the PD-1/PD-L1 axis, which is the target of several therapeutic antibodies, such as nivolumab (anti-PD-1), pembrolizumab (anti-PD-1) and durvalumab (anti-PD-L1), among others (Topalian et al, 2015). Therefore we asked whether smoke exposure affects the expression of PD-L1 and/or PD-1 in our COPD lung tumor model. First we analyzed PD-L1 expression by LLC tumor cells by flow cytometry. LLC cells in culture were mostly PD-L1 positive. Further, analysis of CD45 negative cells from the lungs of tumor bearing mice indicates that smoke exposure before tumor cell injection does not lead to up-regulation of PD-L1 on LLC cells. However, the frequency of PD-L1 positive cells among CD45+ cells was significantly increased in the lungs of tumor bearing mice that had been subjected to cigarette smoke. Smoke exposure also resulted in increased percentages of PD-1+ CD8 and CD4 T cells. It has been noted that in addition to tumor cells themselves monocytes/macrophages can express PD-L1 in the tumor environment (Don et al, 2002; Thompson et al, 2004; Kryczek et al, 2006)). In the mouse model, smoke exposure and tumor challenge did result in an increased frequency of macrophages among lung CD45+ cells. To analyze both the M1 and M2 macrophage sub-populations for PD-L1 expression, cells were first gated on CD11clow/MHCII+, then on CD68+/F480+ macrophages and M1 and M2 populations were distinguished based on iNos and CD206 expression according to Zaynagetdinov et al (2011). M1 and M2 macrophage populations did not reveal significant modulation of PD-L1 expression by either smoke exposure or tumor challenge alone. However, in tumor bearing and smoke exposed mice, lung macrophages are almost exclusively of the M2 phenotype and positive for PD-L1 (PD-L1). The results show that in the lungs of tumor bearing mice exposed to smoke PD-1 as well as PD-L1 expression is increased, and identify M2 macrophages as a significant source of PD-L1 expression in this model, in addition to the LLC tumor cells.

The Anti-Tumor Effect of Anti-PD-L1 Checkpoint Blockade is Enhanced in Smoke Exposed Mice

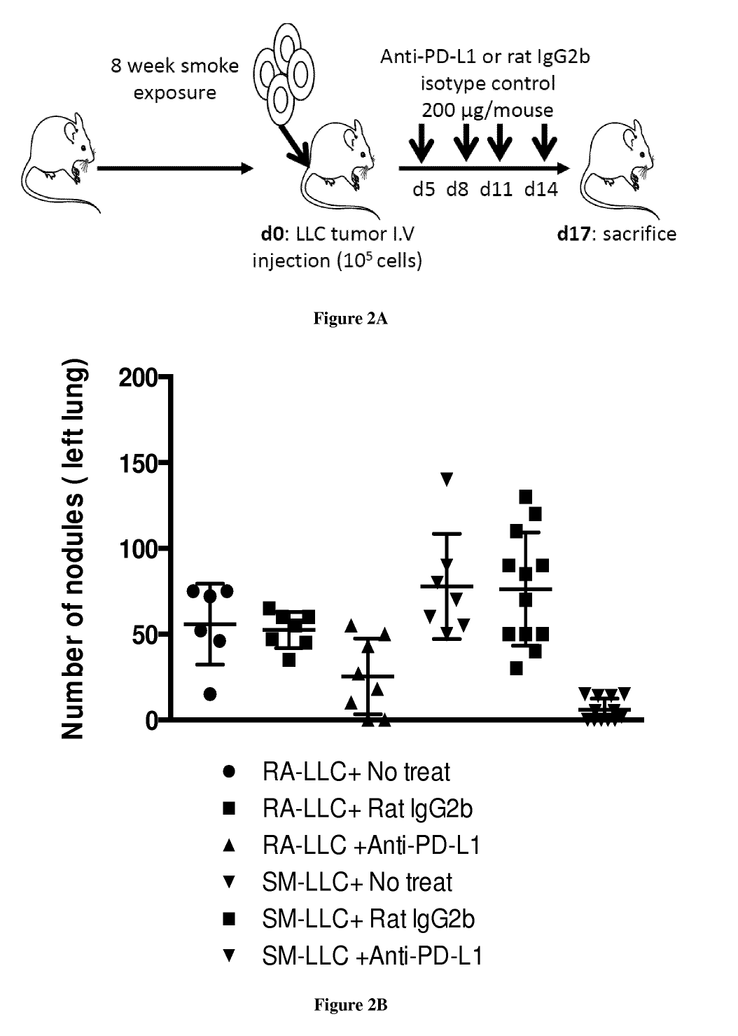

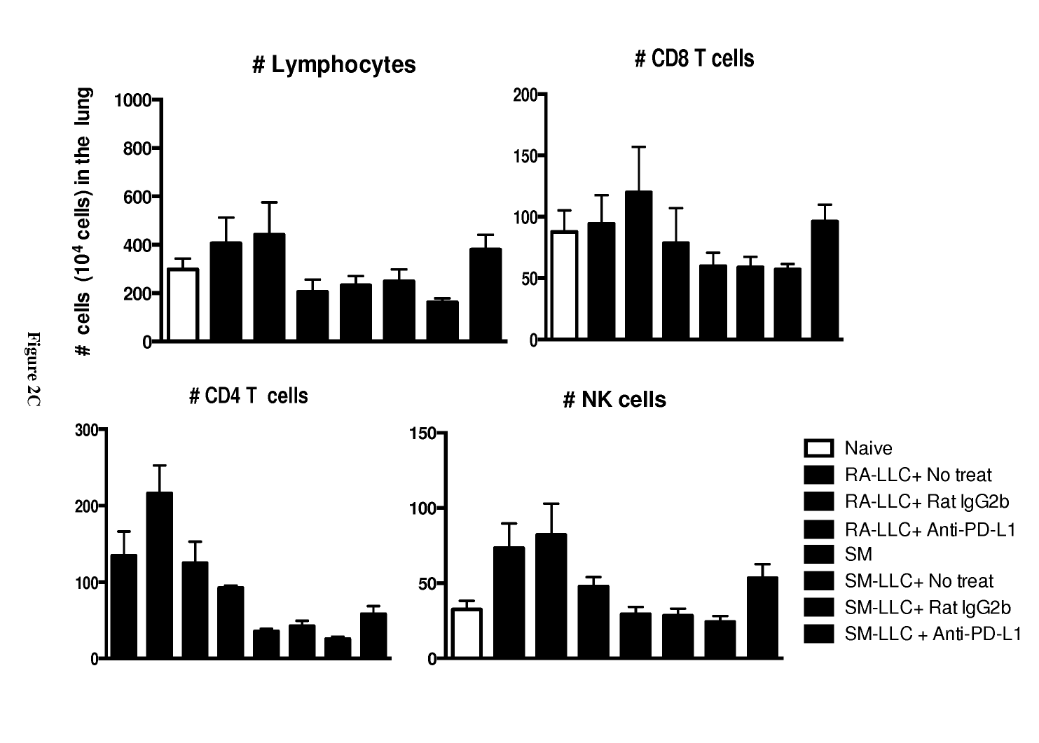

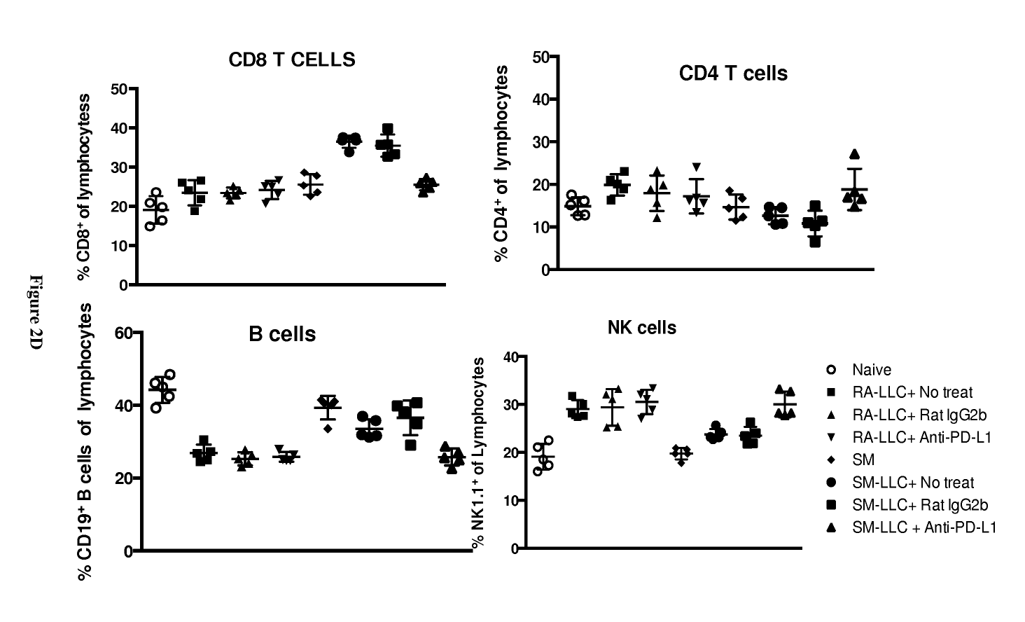

Up-regulation of PD-1/PD-L1, and possibly other checkpoint mechanisms, as a consequence of smoke exposure may be the cause for an ineffective anti-tumor immune response and increased tumor burden as compared to room air exposed mice. To test this hypothesis directly, we subjected mice to 8 weeks of chronic smoke exposure as before. One day after smoke cessation (day 0) tumor cells were injected and on day 5 anti-PD-L1 or isotype control antibody treatment was initiated for a total of four doses (FIG. 2A). Day 5 was selected as treatment start based on preliminary experiments showing that using the pigmented B16 melanoma cell line first clearly visible lung tumor foci can be detected as early as 7 days post i.v. injection of the tumor cells. Thus, the antibody treatment should not interfere with tumor cell trafficking and reflect treatment effects in the lung. As shown in FIG. 2B, anti-PD-L1, but not isotype control antibody, treatment resulted in reduced tumor burden in room air exposed mice. As expected, mice that had been subjected to smoke exposure had significantly greater tumor burden, also when treated with the isotype control antibody. Smoke exposed mice, however, showed a profound response to anti-PD-L1 treatment. Here, anti-PD-L1 treatment had a greater effect than in tumor bearing mice exposed to room air, despite the enhancement of tumor growth by smoke exposure in the control groups. Next, we studied the effect of anti-PD-L1 on lymphocyte distribution in the lung. Although there was a trend towards reduced numbers of lung CD4 T cells and NK cells in smoke exposed mice, the total number of lung lymphocytes was not significantly altered by smoke exposure or tumor challenge (FIG. 2C). Anti-PD-L1 treatment resulted in a partial reversal of reduced CD4 T cell numbers in smoke exposed mice as well as in a trend towards increases in CD8 T cells and NK cells. Anti-PD-L1 treatment did change the distribution of lymphocyte subsets, however, only in mice that had been subjected to smoke exposure (FIG. 2D). Together the results show that anti-PD-L1 checkpoint blockade is more effective in smoke exposed mice and that this effect is associated with changes in lymphocyte distribution, in particular in smoke exposed but not in room air exposed mice treated with anti-PD-L1.

PD-L1 Blockade Improves CD8 and CD4 T Cell and Functionality

We next asked whether anti-PD-L1 treatment resulted in changes in T cell functionality, in particular with regard to expression of IFN-γ, a key Th1 effector cytokine, and expression of Ki67 as a marker for proliferation. Anti-PD-L1 treatment did indeed result in increased frequency of CD8 and CD4 T cells expressing IFN-γ detected by flow cytometry. Up-regulation of IFN-γ as a result of checkpoint blockade could also be detected on the level of mRNA expression by real time quantitative PCR from lung tissue samples. Interestingly, the percentages of Ki67+CD8 and CD4 T cells were only significantly increased upon anti-PD-L1 treatment in smoke exposed mice. These results are consistent with findings by others demonstrating enhanced IFN-γ production with checkpoint blockade, but also point to differences in response to treatment between room air and smoke exposed mice.

Anti-PD-L1 Modulates Expression of Markers of Lymphocyte Activation and Exhaustion

To further assess the effects of anti-PD-L1 on lymphocyte activation we studied the surface expression of selected markers by flow cytometry. As seen before, the percentage of CD69+ NK cells was increased with tumor challenge and further increased with anti-PD-L1 treatment. In smoke exposed mice bearing LLC tumors, CD69+ NK cells were also increased, but to a lesser extent than in room air control mice. Smoke exposure alone also resulted in increases in the percentage of PD-1+ NK cells, an effect which was somewhat more prominent in tumor bearing mice treated with anti-PD-L1 when compared to isotype control treated mice. Increases in CD8 T cells expressing the co-stimulatory receptor ICOS were partially reversed with anti-PD-L1 in smoke exposed mice but remained above the levels found in room air exposed mice. Further, the percentage of CD8 T cells expressing TIM3, another marker of T cell exhaustion, was lower in the anti-PD-L1 treatment group. For CD8 and CD4 T cells, checkpoint blockade reduced the percentage of PD-1+ cells. Interestingly, these effects were only observed in smoke exposed mice, but not in the room air exposed mice treated with anti-PD-L1. Importantly, exposure to chronic cigarette smoke was sufficient for up-regulation of PD-1 on CD8 cells and NK cells and for up-regulation of TIM3 on CD8 T cells. To further explore the functionality of the CD8 and CD4 T cell populations we also assessed how the various treatments might affect the effector/EM compartment. CD8 and CD4 T cells, respectively, were analyzed for expression of CD62L and CD44 and gated for naïve, intermediate, central memory (CM), and the effector/EM cells. The combined analysis from the in vivo experiment with anti-PD-L1 treatment shows an expansion of the CD8 and CD4 effector/EM compartments. This expansion was most pronounced in mice that had been exposed to cigarette smoke.

Effect of PD-L1 Blockade on Immunosuppressive Cells: Macrophages, MDSC and Treg

In addition to expression of co-inhibitory receptors, effector T cell responses can be limited via regulatory CD4 T cells (Treg), which have been found enriched in several tumor types. Importantly, high tumor Treg numbers and low CD8 T cell to Treg ratio have been linked to poor prognosis (Nishikawa and Sakagushi 2010; Nishikawa and Sakaguchi, 2014). Therefore we also investigated how tumor challenge and/or smoke exposure might affect the frequency of lung Treg. Smoke exposure, but not tumor challenge alone, did result in a trend towards increased Treg numbers in lung tissue. However, when analyzed as a percentage of CD4 T cells, smoke exposure resulted in a clear increase in Treg distribution. An increase in Treg upon smoke exposure is also suggested by the increased FoxP3 mRNA expression levels detected in smoke exposed mice when compared to naïve or tumor bearing mice exposed to room air. The change in Treg distribution resulted in a lower CD8 Tcell:Treg ratio in smoke exposed animals. Importantly, in smoke exposed mice, anti-PD-L1 checkpoint blockade significantly reduced the proportion of Treg among CD4 T cells, and increased the CD8 Tcell:Treg ratio. We also investigated the potential effects of anti-PD-L1 on macrophage and myeloid derived suppressor cell (MDSC) populations in the lung. The percentage of macrophages among CD45 cells was increased in tumor bearing mice that had been exposed to chronic cigarette smoke. The relative frequency of macrophages in the lung was unchanged by anti-PD-L1 treatment. Further, checkpoint blockade with anti-PD-L1 did not change the distribution of M1 versus M2 macrophage populations. Due to interference from the treatment anti-PD-L1 antibody we could not assess possible effects on PD-L1 expression by macrophages. While macrophages were largely unaffected by anti-PD-L1 treatment, we did note changes among MDSC populations. Interestingly, the relative frequency of lung granulocytic MDSC was increased in tumor bearing mice exposed to cigarette smoke. This increase was reversed upon treatment with anti-PD-L1. Also for total MDSC anti-PD-L1 treatment normalized the increases seen in tumor bearing mice exposed to cigarette smoke.

### EXAMPLE 4: DISCUSSION

The prognostic value of PD-L1 expression in patients with NSCLC have yielded inconsistent data (57-61). These conflicting results could be attributed to the low statistical power of these studies, to a poor specificity of the different antibodies used, and also to the lack of standardized methods to quantify PD-L1 expression. In our cohort of 435 patients, the bad prognostic value of PD-L1 was strongly related to the density of CD8 T cells in the tumor nests, with a worse survival in the CD8Tu^{high} group of patients, thus reflecting the effectiveness of mechanisms developed by cancers cells to avoid immune surveillance. Rationally, in tumors slightly infiltrated by CD8 T cells, the absence of a prognostic value of PD-L1 was probably linked to the lowest impact of the PD-1/PD-L1 pathway on a weakly active anti-tumor immune response. Most importantly, PD-L1 impact on patients' survival with a high density of CD8Tu cells was even more pronounced in COPD⁺ patients. Moreover, the prognostic value of immune cell densities was completely lost in tumors from COPD patients expressing PD-L1, probably because the level of TIL exhaustion was higher in this group. In NSCLC, anti-PD-1 mAb (Nivolumab) efficacy was shown to increase with the levels of expression of PD-L1 at the tumor cell membrane (62). The results support the rationale that the predictive potential of PD-L1 expression by tumor cells for the response to Nivolumab treatment, should be increased in NSCLC patients with coexisting moderate to severe COPD.

### EXAMPLE 5: HIGH PD-1/TIM-3 CO-EXPRESSION IN COPD PATIENTS ELICITS IMPAIRED ANTI-TUMOR CD8 T CELL RESPONSE AND A SUPERIOR SENSITIVITY TO PD-1 BLOCKADE

COPD does not Affect Immune Cell Density in NSCLC Tumor Microenvironment.

We first investigated the impact of COPD on the composition of the tumor immune microenvironment in a retrospective cohort of NSCLC patients. Among them, 45% had COPD, and in the COPD+ group, 29% had a COPD GOLD stage I (COPD+ I), 60% a COPD GOLD stage II (COPD+ II) and 11% a COPD GOLD stage III (COPD+ III). The mean age, the percentage of male and of smokers were higher for COPD+ than COPD− patients. Coexisting COPD was associated with worse survival only for NSCLC stage I patients. Because of the small number of COPD+ III patients, the COPD+ II and COPD+ III groups were merged for most of the subsequent analyses. COPD+ and COPD− patients did not differ in density of neutrophils (CD66b+ cells), macrophages (CD68+ cells), mature DCs (DC-Lamp+ cells) and CD8 T cells in tumor nests (CD8Tu) and in stroma (CD8s), regardless of GOLD stage.

COPD is Associated to a Down-Regulation of Gene Expression Related to Type I IFN Response and to T Cell Pathways in NSCLC Tumor Microenvironment.

From 52 NSCLC patients, we compared gene expression profile related to the immune response in cancer, according to patients' COPD status. In this cohort, 20 patients had a COPD, and among them, 35% had a COPD+ I and 65% a COPD+ II-III. 34 genes were down-regulated in the COPD+ group (fold change >1.5 and p<0.01). Interestingly, none of these genes were related to a particular immune cell population (CDR, CD4, CD8a, FoxP3, CD19, NCR1, NCAM1, CD68, CD163 and CEACAM8), and the only down-regulated gene with a fold change >1.5 was CD8a (p=0.02). Moreover, the down-regulated genes were associated to two main pathways, type I IFN response (IRF4, IRF5 and IRF8) and a pathway related to T cells. The T cells associated pathway was the one which involved most genes, and identified in patients with COPD a down-regulation of genes associated to alpha-beta T cell selection, differentiation and activation. Interestingly, the most down-regulated gene in this pathway was ZAP70 (Fold change=1.95), a tyrosine kinase involved in the initial step of TCR-mediated signal transduction. In agreement, using a more stringent method (fold change>1.5 and FDR<0.1), genes down-regulated in the COPD+ group were still associated to type I IFN response and to T cell related pathways.

TIL Exhaustion, Identified by PD-1/TIM-3 Co-Expression, is Correlated with COPD Severity

According to above results, we investigated whether effector functions of TILs were altered in COPD+ patients using a prospective cohort of 50 NSCLC patients. We first identified, that within the tumor tissue (Tumor), the proportion of CTLA-4+, LAG-3+, PD-1+ and TIM-3+ cells among CD4 and CD8 T cells was systematically higher than in the other anatomical sites (blood and non-tumor distal lung tissue (NT)), regardless of COPD status. Regarding effector molecule or cytokine secretion, the frequency of CD4 and CD8 T cells positive for Granzyme B, TNF-α, IFN-γ and IL-17 was significantly lower in Tumor than in NT. We also investigated the proportion of regulatory CD4 T cells (Treg, CD4+ FoxP3+), and found a marked increased of FoxP3+ cell frequency among CD4 T cells in Tumor.

Concerning CD8 TILs, PD-1 and TIM-3 expression was strongly positively correlated, as was frequency of IFN-γ+ and TNF-α+ cells. Moreover, both TNF-α and IFN-γ secretion was inversely associated with TIM-3 expression, while frequency of PD-1+ cells was only inversely linked to TNF-α secretion. Remarkably, CD8 TILs co-expressing PD-1 and TIM-3 were restricted to Tumor, and this cell subtype was the only one highly inversely correlated with both IFN-γ and TNF-α secretion. Since two different flow cytometry panels were used to study cytokine secretion and immune-checkpoint expression, a third one was designed to analyze PD-1/TIM-3 expression and cytokine production in the same cells. In tumors from 10 patients, this strategy confirmed that CD8 TILs co-expressing PD-1/TIM-3 had the lowest secretion of TNF-α and IFN-γ. Overall, similar results were observed regarding CD4 TILs. However, less CD4 TILs coexpressed PD-1/TIM-3, and the relationship between cytokine secretion and PD-1/TIM-3 coexpression was weaker.

Based on above results, we investigated the link between COPD and TIL exhaustion. In COPD, airflow obstruction severity is inversely correlated with the Forced Expiratory Volume in 1 second expressed as a percentage of normal predicted values (FEV1% predicted) (see Method section). Remarkably, FEV1% predicted was inversely correlated with the proportion of CD8 TILs expressing PD-1 and co-expressing PD-1/TIM-3 in COPD+ patients only. In agreement, in COPD+ patients, FEV1% predicted was positively correlated with the proportion of CD8 TILs secreting TNF-α and IFN-γ. Regarding CD4 TILs, only IFN-γ was positively correlated with FEV1% predicted. Interestingly, frequency of TIM-3+, PD-1+ and TIM-3+/PD-1+ cells among CD4 and CD8 TILs was higher in COPD+ II-III patients than in COPD− patients. Moreover, the proportion of Treg among CD4 TILs was not different according to patients' COPD status and Gold stages. Overall, this set of experiments shown that COPD severity was strongly correlated with TIL exhaustion, and that this association was more pronounced for CD8 TILs.

Strong Correlation Between CD8Tu Cell Density and CD8 TIL Exhaustion: A Phenomenon Exacerbated in COPD+ Patients

An association between CD8 TIL exhaustion (PD-1+ cell frequency) and the immune composition of the tumor microenvironment (density of CD3+, CD8 and CD45RO+ T cells) was recently reported in colorectal cancer (66). We investigated this interrelation in our prospective cohort and then studied the impact of COPD. First, CD8 TIL exhaustion and cytokine secretion were only linked to CD8Tu cell and CD8S cell density. Regarding CD4 TILs, none of the immune cell densities studied was associated with their exhaustion, and only their cytokine secretion was slightly inversely correlated with CD8S cell density. Due to the strong association between CD8 TIL exhaustion and their density in the tumor nests, we then focused our analysis on CD8 TILs. Secondly, CD8Tu cell density and CD8 TIL exhaustion were more strongly associated in COPD+ patients than in COPD− patients.

To confirm these results, median CD8Tu cell density was used to separate patients into two groups according to a Low or a High CD8Tu cell density. In the CD8TuLow group, the level of CD8 TIL exhaustion did not differ according to COPD status. In the CD8TuHigh group, the frequencies of CD8 TILs expressing TIM-3 and co-expressing PD-1/TIM-3 were higher in COPD+ patients than in COPD− patients. Overall, CD8 TIL exhaustion was restricted to highly CD8 T cell infiltrated tumors and this phenomenon was exacerbated in COPD+ patients.

Tumor Immune Profiles are Differentially Orchestrated in COPD+ Patients

The strong impact of immunosuppression on tumor burden is based on TIL exhaustion, but also on concomitant mechanisms that malignant cells develop to avoid immune surveillance. The most-studied mechanism is probably PD-L1 expression by malignant cells. No difference of PD-L1 expression by tumor cells was observed according to patients' COPD status and Gold stages (retrospective cohort). In various cancer types, high CD8 T cell density is associated with high PD-L1 expression by tumor cells (54, 67). Whatever the COPD status of the patients, we confirmed this result. Moreover, the frequency of tumor cells expressing PD-L1 was also higher, but to a lesser extent, in neutrophilHigh, macrophageHigh and DC-LampHigh groups. Additionally, among these highly infiltrated groups, PD-L1 expression was similar between COPD− and COPD⁺ patients.

We then investigated the link between PD-L1 expression by tumor cells and CD8 TIL exhaustion (prospective cohort). In contrast to COPD− patients, in COPD+ patients, the frequency of PD-1+ TIM-3+ cells among CD8 TILs was positively correlated with frequency of PD-L1+ tumor cells. In COPD− patients, CD8Tu cell, CD8s cell, DC-Lamp+ cell, macrophage densities and frequency of PD-L1+ tumor cells were part of the same cluster (cluster-1a) and did not cluster directly with PD-1 and TIM-3 expression by CD8 TILs (cluster-2a). In COPD+ patients, CD8Tu cell and CD8S cell densities were directly associated with the frequency of CD8 TILs expressing PD-1 and TIM-3, and with PD-L1 expression by tumor cells (cluster-1b). In contrast to COPD− patients, in COPD+ patients, DC-Lamp+ cell density did not cluster directly with CD8 T cell densities. Altogether, in COPD+ patients, an immune reactive milieu, characterized by a high CD8 T cell density, seemed to be strongly associated with an immune profile that would be less efficient in counteracting the tumor burden.

Absence of Immune Cell Prognostic Value in NSCLC Patients with Moderate to Severe COPD

We investigated whether the disturbed tumor immune microenvironment identified in patients with COPD, was associated with an altered impact of immune cell densities on patient survival. In the whole retrospective cohort and in the COPD− group, univariate Cox-regression analysis showed that CD8Tu, CD8s and DC-Lamp+ cell densities were all associated with favorable prognostic value, while neutrophil and macrophage density had no impact on patient survival. In COPD+ patients, CD8Tu cell density did not affect patient survival. Furthermore, CD8Tu, CD8S and DC-Lamp+ cell densities were not significantly associated with improved survival in COPD+ II-III patients.

Then, patients were stratified by quartiles of CD8Tu cell density. In COPD− patients, CD8Tu cell density was associated with longer overall survival (OS) as soon as the 2nd quartile was reached, whereas in COPD+ and COPD+ II-III patients the survival curves for all quartiles merged together. DC-Lamp+ cell and CD8S cell densities were not associated with significant prognostic value only in COPD+ II-III patients. Multivariate Cox-regression analysis adjusted for age, gender, vascular emboli, smoking history and stratified on tumor stages, highlighted the absence of CD8 T cell prognostic value for the stroma and tumor nests in COPD+ patients. Together, these data might suggest that the protective impact of a high adaptive immune cell infiltration in NSCLC is altered in COPD+ patients and identify CD8Tu cells as the most affected population.

PD-L1 Expression by Malignant Cells is Associated with Shorter Survival Only in CD8TuHigh COPD+ Patients

From the above results, we investigated whether coexisting COPD modified the prognostic value of PD-L1. Whatever the group of patients considered, PD-L1 expression was not associated with significant prognostic value. Since PD-L1 expression was strongly linked to CD8 T cell density, we then deciphered the prognostic value of PD-L1 according to a High/Low CD8Tu cell density, and also to patient COPD status. For CD8TuLow groups, PD-L1 expression was not associated with significant prognostic value in COPD− or in COPD+ patients. Interestingly, for CD8TuHigh groups, PD-L1 expression did not affect survival for COPD− patients, but was associated with a reduced OS for COPD+ patients. Moreover, in COPD− patients, the prognostic value of CD8Tu and of CD8S cell density was similar whether tumor cells expressed PD-L1 or not. Remarkably, for COPD+ patients, CD8Tu and CD8S cell densities were associated with extended OS for those with PD-L1− tumors, while these prognostic values were not observed in the PD-L1+ groups. Finally, these results were confirmed in subgroups of patients defined by a cut-off of PD-L1+ tumor cell frequency ≥5% and ≥10%.

Anti-PD-1 Antibody (Nivolumab) Efficacy in Advanced-Stage NSCLC Patients According to a Coexisting COPD and to Smoke Exposure

We investigated the impact of COPD on the effectiveness of an anti-PD-1 antibody by using a cohort of 34 patients with advanced-stage NSCLC receiving nivolumab. At the completion of the study, a higher percentage of patients with an ongoing response and a significant longer progression-free survival (PFS) were observed in the COPD+ group. However, it was previously shown that the efficacy of pembrolizumab, another anti-PD-1 antibody, was greater in patients with a smoking-associated mutational signature (21). Consequently, we investigated whether the better efficacy of nivolumab in COPD+ patients was linked to the higher smoke exposure observed in this group. In the non-COPD group, the responders were heterogeneously distributed according to their smoking exposure (number of pack-years) and among the five non-smoker patients, three of them had a response to nivolumab. In COPD+ patients, not all heavy smokers responded to nivolumab, but most “long” responder-patients were heavy smokers (smoke exposure comprised between 40 and 60 pack-years). Moreover, in the whole cohort, a smoke exposure >30 or >40 pack-years was associated with a better PFS, while in non-COPD patients, smoke exposure (Cut-off: >5, >20, >30 pack-years) was not associated with a better PFS or OS. Remarkably, in the COPD+ group, a smoke exposure >30 pack-years was associated with a better PFS and also with a dramatic improvement of OS.

Finally, we investigated in our retrospective cohort of 435 NSCLC patients, whether immune cell prognostic value (CD8Tu, CD8S and DC-Lamp+ cells) was impacted by a strong smoke exposure (>30 pack-years). In heavy smokers, immune cell prognostic value was stronger in non-COPD patients. Inversely, in COPD+ patients, CD8S cell prognostic value was not significant in heavy smokers, and the one of CD8Tu cells was completely absent (HR:1.01, p=0.948). Altogether, our results might suggest a higher efficacy of nivolumab in NSCLC patients with COPD, a group characterized by a complete loss of CD8 T cell-associated favorable clinical outcome in heavy smokers.

### REFERENCES

Throughout this application, various references describe the state of the art to which this invention pertains. The disclosures of these references are hereby incorporated by reference into the present disclosure.
1. J. Galon, A. Costes, F. Sanchez-Cabo, A. Kirilovsky, B. Mlecnik, C. Lagorce-Pages, M. Tosolini, M. Camus, A. Berger, P. Wind, F. Zinzindohoué, P. Bruneval, P.-H. Cugnenc, Z. Trajanoski, W.-H. Fridman, F. Pages, Type, density, and location of immune cells within human colorectal tumors predict clinical outcome, Science 313, 1960-1964 (2006).
2. S. M. A. Mahmoud, E. C. Paish, D. G. Powe, R. D. Macmillan, M. J. Grainge, A. H. S. Lee, I. O. Ellis, A. R. Green, Tumor-infiltrating CD8+ lymphocytes predict clinical outcome in breast cancer, J. Clin. Oncol. Off. J. Am. Soc. Clin. Oncol. 29, 1949-1955 (2011).
3. K. I. Al-Shibli, T. Donnem, S. Al-Saad, M. Persson, R. M. Bremnes, L.-T. Busund, Prognostic effect of epithelial and stromal lymphocyte infiltration in non-small cell lung cancer, Clin. Cancer Res. Off. J. Am. Assoc. Cancer Res. 14, 5220-5227 (2008).
4. F. Azimi, R. A. Scolyer, P. Rumcheva, M. Moncrieff, R. Murali, S. W. McCarthy, R. P. Saw, J. F. Thompson, Tumor-infiltrating lymphocyte grade is an independent predictor of sentinel lymph node status and survival in patients with cutaneous melanoma, J. Clin. Oncol. Off. J. Am. Soc. Clin. Oncol. 30, 2678-2683 (2012).
5. L. Zhang, J. R. Conejo-Garcia, D. Katsaros, P. A. Gimotty, M. Massobrio, G. Regnani, A. Makrigiannakis, H. Gray, K. Schlienger, M. N. Liebman, S. C. Rubin, G. Coukos, Intratumoral T cells, recurrence, and survival in epithelial ovarian cancer, N. Engl. J. Med. 348, 203-213 (2003).
6. P. Sharma, Y. Shen, S. Wen, S. Yamada, A. A. Jungbluth, S. Gnjatic, D. F. Bajorin, V. E. Reuter, H. Herr, L. J. Old, E. Sato, CD8 tumor-infiltrating lymphocytes are predictive of survival in muscle-invasive urothelial carcinoma, Proc. Natl. Acad. Sci. U.S.A. 104, 3967-3972 (2007).
7. F. Pagès, J. Galon, M.-C. Dieu-Nosjean, E. Tartour, C. Sautès-Fridman, W.-H. Fridman, Immune infiltration in human tumors: a prognostic factor that should not be ignored, Oncogene 29, 1093-1102 (2010).
8. F. Pages, A. Kirilovsky, B. Mlecnik, M. Asslaber, M. Tosolini, G. Bindea, C. Lagorce, P. Wind, F. Marliot, P. Bruneval, K. Zatloukal, Z. Trajanoski, A. Berger, W.-H. Fridman, J. Galon, In situ cytotoxic and memory T cells predict outcome in patients with early-stage colorectal cancer, J. Clin. Oncol. Off. J. Am. Soc. Clin. Oncol. 27, 5944-5951 (2009).
9. W. H. Fridman, F. Pages, C. Sautès-Fridman, J. Galon, The immune contexture in human tumours: impact on clinical outcome, Nat. Rev. Cancer 12, 298-306 (2012).
10. M.-C. Dieu-Nosjean, J. Goc, N. A. Giraldo, C. Sautès-Fridman, W. H. Fridman, Tertiary lymphoid structures in cancer and beyond, Trends Immunol. 35, 571-580 (2014).
11. C. Germain, S. Gnjatic, M.-C. Dieu-Nosjean, Tertiary Lymphoid Structure-Associated B Cells are Key Players in Anti-Tumor Immunity, Front. Immunol. 6, 67 (2015).
12. A. Ladányi, J. Kiss, B. Somlai, K. Gilde, Z. Fejos, A. Mohos, I. Gaudi, J. Tímár, Density of DC-LAMP(+) mature dendritic cells in combination with activated T lymphocytes infiltrating primary cutaneous melanoma is a strong independent prognostic factor, Cancer Immunol. Immunother. CII 56, 1459-1469 (2007).
13. L. Martinet, I. Garrido, T. Filleron, S. Le Guellec, E. Bellard, J.-J. Fournie, P. Rochaix, J.-P. Girard, Human solid tumors contain high endothelial venules: association with T- and B-lymphocyte infiltration and favorable prognosis in breast cancer, Cancer Res. 71, 5678-5687 (2011).
14. J. Goc, C. Germain, T. K. D. Vo-Bourgais, A. Lupo, C. Klein, S. Knockaert, L. de Chaisemartin, H. Ouakrim, E. Becht, M. Alifano, P. Validire, R. Remark, S. A. Hammond, I. Cremer, D. Damotte, W.-H. Fridman, C. Sautès-Fridman, M.-C. Dieu-Nosjean, Dendritic cells in tumor-associated tertiary lymphoid structures signal a Th1 cytotoxic immune contexture and license the positive prognostic value of infiltrating CD8+ T cells, Cancer Res. 74, 705-715 (2014).
15. O. Hamid, C. Robert, A. Daud, F. S. Hodi, W.-J. Hwu, R. Kefford, J. D. Wolchok, P. Hersey, R. W. Joseph, J. S. Weber, R. Dronca, T. C. Gangadhar, A. Patnaik, H. Zarour, A. M. Joshua, K. Gergich, J. Elassaiss-Schaap, A. Algazi, C. Mateus, P. Boasberg, P. C. Tumeh, B. Chmielowski, S. W. Ebbinghaus, X. N. Li, S. P. Kang, A. Ribas, Safety and tumor responses with lambrolizumab (anti-PD-1) in melanoma, N. Engl. J. Med. 369, 134-144 (2013).
16. S. L. Topalian, F. S. Hodi, J. R. Brahmer, S. N. Gettinger, D. C. Smith, D. F. McDermott, J. D. Powderly, R. D. Carvajal, J. A. Sosman, M. B. Atkins, P. D. Leming, D. R. Spigel, S. J. Antonia, L. Horn, C. G. Drake, D. M. Pardoll, L. Chen, W. H. Sharfman, R. A. Anders, J. M. Taube, T. L. McMiller, H. Xu, A. J. Korman, M. Jure-Kunkel, S. Agrawal, D. McDonald, G. D. Kollia, A. Gupta, J. M. Wigginton, M. Sznol, Safety, activity, and immune correlates of anti-PD-1 antibody in cancer, N. Engl. J. Med. 366, 2443-2454 (2012).
17. J. R. Brahmer, S. S. Tykodi, L. Q. M. Chow, W.-J. Hwu, S. L. Topalian, P. Hwu, C. G. Drake, L. H. Camacho, J. Kauh, K. Odunsi, H. C. Pitot, O. Hamid, S. Bhatia, R. Martins, K. Eaton, S. Chen, T. M. Salay, S. Alaparthy, J. F. Grosso, A. J. Korman, S. M. Parker, S. Agrawal, S. M. Goldberg, D. M. Pardoll, A. Gupta, J. M. Wigginton, Safety and activity of anti-PD-L1 antibody in patients with advanced cancer, N. Engl. J. Med. 366, 2455-2465 (2012).
18. R. J. Motzer, B. I. Rini, D. F. McDermott, B. G. Redman, T. M. Kuzel, M. R. Harrison, U. N. Vaishampayan, H. A. Drabkin, S. George, T. F. Logan, K. A. Margolin, E. R. Plimack, A. M. Lambert, I. M. Waxman, H. J. Hammers, Nivolumab for Metastatic Renal Cell Carcinoma: Results of a Randomized Phase II Trial, J. Clin. Oncol. Off. J. Am. Soc. Clin. Oncol. 33, 1430-1437 (2015).
19. S. M. Ansell, A. M. Lesokhin, I. Borrello, A. Halwani, E. C. Scott, M. Gutierrez, S. J. Schuster, M. M. Millenson, D. Cattry, G. J. Freeman, S. J. Rodig, B. Chapuy, A. H. Ligon, L. Zhu, J. F. Grosso, S. Y. Kim, J. M. Timmerman, M. A. Shipp, P. Armand, PD-1 blockade with nivolumab in relapsed or refractory Hodgkin's lymphoma, N. Engl. J. Med. 372, 311-319 (2015).
20. T. Powles, J. P. Eder, G. D. Fine, F. S. Braiteh, Y. Loriot, C. Cruz, J. Bellmunt, H. A. Burris, D. P. Petrylak, S. Teng, X. Shen, Z. Boyd, P. S. Hegde, D. S. Chen, N. J. Vogelzang, MPDL3280A (anti-PD-L1) treatment leads to clinical activity in metastatic bladder cancer, Nature 515, 558-562 (2014).
21. A. M. M. Eggermont, V. Chiarion-Sileni, J.-J. Grob, R. Dummer, J. D. Wolchok, H. Schmidt, O. Hamid, C. Robert, P. A. Ascierto, J. M. Richards, C. Lebbé, V. Ferraresi, M. Smylie, J. S. Weber, M. Maio, C. Konto, A. Hoos, V. de Pril, R. K. Gurunath, G. de Schaetzen, S. Suciu, A. Testori, Adjuvant ipilimumab versus placebo after complete resection of high-risk stage III melanoma (EORTC 18071): a randomised, double-blind, phase 3 trial, Lancet Oncol. 16, 522-530 (2015).
22. F. Balkwill, A. Mantovani, Inflammation and cancer: back to Virchow?, Lancet Lond. Engl. 357, 539-545 (2001).
23. N. R. Salama, M. L. Hartung, A. Müller, Life in the human stomach: persistence strategies of the bacterial pathogen Helicobacter pylori, Nat. Rev. Microbiol. 11, 385-399 (2013).
24. E. Bessède, C. Staedel, L. A. Acuña Amador, P. H. Nguyen, L. Chambonnier, M. Hatakeyama, G. Belleannée, F. Mégraud, C. Varon, Helicobacter pylori generates cells with cancer stem cell properties via epithelial-mesenchymal transition-like changes, Oncogene 33, 4123-4131 (2014).
25. M. Schiffman, P. E. Castle, J. Jeronimo, A. C. Rodriguez, S. Wacholder, Human papillomavirus and cervical cancer, Lancet Lond. Engl. 370, 890-907 (2007).
26. A. Arzumanyan, H. M. G. P. V. Reis, M. A. Feitelson, Pathogenic mechanisms in HBV- and HCV-associated hepatocellular carcinoma, Nat. Rev. Cancer 13, 123-135 (2013).
27. T. Jess, E. V. Loftus, F. S. Velayos, W. S. Harmsen, A. R. Zinsmeister, T. C. Smyrk, C. D. Schleck, W. J. Tremaine, L. J. Melton, P. Munkholm, W. J. Sandborn, Risk of intestinal cancer in inflammatory bowel disease: a population-based study from olmsted county, Minnesota, Gastroenterology 130, 1039-1046 (2006).
28. E. R. Kim, D. K. Chang, Colorectal cancer in inflammatory bowel disease: the risk, pathogenesis, prevention and diagnosis, World J. Gastroenterol. 20, 9872-9881 (2014).
29. A. V. Pinho, L. Chantrill, I. Rooman, Chronic pancreatitis: A path to pancreatic cancer, Cancer Lett. 345, 203-209 (2014).
30. A. Mantovani, P. Allavena, A. Sica, F. Balkwill, Cancer-related inflammation, Nature 454, 436-444 (2008).
31. N. A. Giraldo, E. Becht, Y. Vano, C. Sautès-Fridman, W. H. Fridman, The immune response in cancer: from immunology to pathology to immunotherapy, Virchows Arch. Int. J. Pathol. 467, 127-135 (2015).
32. B.-Z. Qian, J. W. Pollard, Macrophage diversity enhances tumor progression and metastasis, Cell 141, 39-51 (2010).
33. WHO|The top 10 causes of death (available at http://www.who.int/mediacentre/factsheets/fs310/en/).
34. J. P. de Torres, J. M. Marín, C. Casanova, C. Cote, S. Carrizo, E. Cordoba-Lanus, R. Baz-Dávila, J. J. Zulueta, A. Aguirre-Jaime, M. Saetta, M. G. Cosio, B. R. Celli, Lung cancer in patients with chronic obstructive pulmonary disease—incidence and predicting factors, Am. J. Respir. Crit. Care Med. 184, 913-919 (2011).
35. G. G. Brusselle, G. F. Joos, K. R. Bracke, New insights into the immunology of chronic obstructive pulmonary disease, Lancet Lond. Engl. 378, 1015-1026 (2011).
36. A. M. Houghton, Mechanistic links between COPD and lung cancer, Nat. Rev. Cancer 13, 233-245 (2013).
37. R. P. Young, R. J. Hopkins, T. Christmas, P. N. Black, P. Metcalf, G. D. Gamble, COPD prevalence is increased in lung cancer, independent of age, sex and smoking history, Eur. Respir. J. 34, 380-386 (2009).
38. P. Qu, J. Roberts, Y. Li, M. Albrecht, 0. W. Cummings, J. N. Eble, H. Du, C. Yan, Stat3 downstream genes serve as biomarkers in human lung carcinomas and chronic obstructive pulmonary disease, Lung Cancer 63, 341-347 (2009).
39. R. Zhai, X. Yu, A. Shafer, J. C. Wain, D. C. Christiani, The impact of coexisting COPD on survival of patients with early-stage non-small cell lung cancer undergoing surgical resection, Chest 145, 346-353 (2014).
40. M. C. Turner, Y. Chen, D. Krewski, E. E. Calle, M. J. Thun, Chronic obstructive pulmonary disease is associated with lung cancer mortality in a prospective study of never smokers, Am. J. Respir. Crit. Care Med. 176, 285-290 (2007).
41. K. Hiraoka, M. Miyamoto, Y. Cho, M. Suzuoki, T. Oshikiri, Y. Nakakubo, T. Itoh, T. Ohbuchi, S. Kondo, H. Katoh, Concurrent infiltration by CD8+ T cells and CD4+ T cells is a favourable prognostic factor in non-small-cell lung carcinoma, Br. J. Cancer 94, 275-280 (2006).
42. P. Maby, D. Tougeron, M. Hamieh, B. Mlecnik, H. Kora, G. Bindea, H. K. Angell, T. Fredriksen, N. Elie, E. Fauquembergue, A. Drouet, J. Leprince, J. Benichou, J. Mauillon, F. Le Pessot, R. Sesboüé, J.-J. Tuech, J.-C. Sabourin, P. Michel, T. Frébourg, J. Galon, J.-B. Latouche, Correlation between Density of CD8+ T-cell Infiltrate in Microsatellite Unstable Colorectal Cancers and Frameshift Mutations: A Rationale for Personalized Immunotherapy, Cancer Res. 75, 3446-3455 (2015).
43. N. J. Llosa, M. Cruise, A. Tam, E. C. Wicks, E. M. Hechenbleikner, J. M. Taube, R. L. Blosser, H. Fan, H. Wang, B. S. Luber, M. Zhang, N. Papadopoulos, K. W. Kinzler, B. Vogelstein, C. L. Sears, R. A. Anders, D. M. Pardoll, F. Housseau, The vigorous immune microenvironment of microsatellite instable colon cancer is balanced by multiple counter-inhibitory checkpoints, Cancer Discov. 5, 43-51 (2015).
44. J. Fourcade, Z. Sun, M. Benallaoua, P. Guillaume, I. F. Luescher, C. Sander, J. M. Kirkwood, V. Kuchroo, H. M. Zarour, Upregulation of Tim-3 and PD-1 expression is associated with tumor antigen-specific CD8+ T cell dysfunction in melanoma patients, J. Exp. Med. 207, 2175-2186 (2010).
45. A. Gros, P. F. Robbins, X. Yao, Y. F. Li, S. Turcotte, E. Tran, J. R. Wunderlich, A. Mixon, S. Farid, M. E. Dudley, K.-I. Hanada, J. R. Almeida, S. Darko, D. C. Douek, J. C. Yang, S. A. Rosenberg, PD-1 identifies the patient-specific CD8⁺ tumor-reactive repertoire infiltrating human tumors, J. Clin. Invest. 124, 2246-2259 (2014).
46. D. S. Thommen, J. Schreiner, P. Müller, P. Herzig, A. Roller, A. Belousov, P. Umana, P. Pisa, C. Klein, M. Bacac, O. S. Fischer, W. Moersig, S. Savic Prince, V. Levitsky, V. Karanikas, D. Lardinois, A. Zippelius, Progression of Lung Cancer Is Associated with Increased Dysfunction of T Cells Defined by Coexpression of Multiple Inhibitory Receptors, Cancer Immunol. Res. 3, 1344-1355 (2015).
47. Y. Agata, A. Kawasaki, H. Nishimura, Y. Ishida, T. Tsubata, H. Yagita, T. Honjo, Expression of the PD-1 antigen on the surface of stimulated mouse T and B lymphocytes, Int. Immunol. 8, 765-772 (1996).
48. J. W. Austin, P. Lu, P. Majumder, R. Ahmed, J. M. Boss, STAT3, STAT4, NFATc1, and CTCF regulate PD-1 through multiple novel regulatory regions in murine T cells, J. Immunol. Baltim. Md 1950 192, 4876-4886 (2014).
49. A. Bhowmik, T. A. Seemungal, R. J. Sapsford, J. A. Wedzicha, Relation of sputum inflammatory markers to symptoms and lung function changes in COPD exacerbations, Thorax 55, 114-120 (2000).
50. S. Grumelli, D. B. Corry, L.-Z. Song, L. Song, L. Green, J. Huh, J. Hacken, R. Espada, R. Bag, D. E. Lewis, F. Kheradmand, An immune basis for lung parenchymal destruction in chronic obstructive pulmonary disease and emphysema, PLoS Med. 1, e8 (2004).
51. N. A. Giraldo, E. Becht, R. Remark, D. Damotte, C. Sautès-Fridman, W. H. Fridman, The immune contexture of primary and metastatic human tumours, Curr. Opin. Immunol. 27, 8-15 (2014).
52. C. M. Kinsey, R. San José Estépar, Y. Wei, G. R. Washko, D. C. Christiani, Regional Emphysema of a Non-Small Cell Tumor Is Associated with Larger Tumors and Decreased Survival Rates, Ann. Am. Thorac. Soc. 12, 1197-1205 (2015).
53. J. M. Taube, R. A. Anders, G. D. Young, H. Xu, R. Sharma, T. L. McMiller, S. Chen, A. P. Klein, D. M. Pardoll, S. L. Topalian, L. Chen, Colocalization of inflammatory response with B7-hl expression in human melanocytic lesions supports an adaptive resistance mechanism of immune escape, Sci. Transl. Med. 4, 127ra37 (2012).
54. P. C. Tumeh, C. L. Harview, J. H. Yearley, I. P. Shintaku, E. J. M. Taylor, L. Robert, B. Chmielowski, M. Spasic, G. Henry, V. Ciobanu, A. N. West, M. Carmona, C. Kivork, E. Seja, G. Cherry, A. J. Gutierrez, T. R. Grogan, C. Mateus, G. Tomasic, J. A. Glaspy, R. O. Emerson, H. Robins, R. H. Pierce, D. A. Elashoff, C. Robert, A. Ribas, PD-1 blockade induces responses by inhibiting adaptive immune resistance, Nature 515, 568-571 (2014).
55. C. Xu, C. M. Fillmore, S. Koyama, H. Wu, Y. Zhao, Z. Chen, G. S. Herter-Sprie, E. A. Akbay, J. H. Tchaicha, A. Altabef, J. B. Reibel, Z. Walton, H. Ji, H. Watanabe, P. A. Jänne, D. H. Castrillon, A. K. Rustgi, A. J. Bass, G. J. Freeman, R. F. Padera, G. Dranoff, P. S. Hammerman, C. F. Kim, K.-K. Wong, Loss of Lkbl and Pten leads to lung squamous cell carcinoma with elevated PD-L1 expression, Cancer Cell 25, 590-604 (2014).
56. A. T. Parsa, J. S. Waldron, A. Panner, C. A. Crane, I. F. Parney, J. J. Barry, K. E. Cachola, J. C. Murray, T. Tihan, M. C. Jensen, P. S. Mischel, D. Stokoe, R. O. Pieper, Loss of tumor suppressor PTEN function increases B7-H1 expression and immunoresistance in glioma, Nat. Med. 13, 84-88 (2007).
57. L. H. Schmidt, A. Kümmel, D. Görlich, M. Mohr, S. Bröckling, J. H. Mikesch, I. Grünewald, A. Marra, A. M. Schultheis, E. Wardelmann, C. Müller-Tidow, T. Spieker, C. Schliemann, W. E. Berdel, R. Wiewrodt, W. Hartmann, PD-1 and PD-L1 Expression in NSCLC Indicate a Favorable Prognosis in Defined Subgroups, PLoS ONE 10, e0136023 (2015).
58. Y. Chen, C.-Y. Mu, J.-A. Huang, Clinical significance of programmed death-1 ligand-1 expression in patients with non-small cell lung cancer: a 5-year-follow-up study, Tumori 98, 751-755 (2012).
59. W. A. Cooper, T. Tran, R. E. Vilain, J. Madore, C. I. Selinger, M. Kohonen-Corish, P. Yip, B. Yu, S. A. O'Toole, B. C. McCaughan, J. H. Yearley, L. G. Horvath, S. Kao, M. Boyer, R. A. Scolyer, PD-L1 expression is a favorable prognostic factor in early stage non-small cell carcinoma, Lung Cancer Amst. Neth. 89, 181-188 (2015).
60. V. Velcheti, K. A. Schalper, D. E. Carvajal, V. K. Anagnostou, K. N. Syrigos, M. Sznol, R. S. Herbst, S. N. Gettinger, L. Chen, D. L. Rimm, Programmed death ligand-1 expression in non-small cell lung cancer, Lab. Investig. J. Tech. Methods Pathol. 94, 107-116 (2014).
61. C.-Y. Mu, J.-A. Huang, Y. Chen, C. Chen, X.-G. Zhang, High expression of PD-L1 in lung cancer may contribute to poor prognosis and tumor cells immune escape through suppressing tumor infiltrating dendritic cells maturation, Med. Oncol. Northwood Lond. Engl. 28, 682-688 (2011).
62. H. Borghaei, L. Paz-Ares, L. Horn, D. R. Spigel, M. Steins, N. E. Ready, L. Q. Chow, E. E. Vokes, E. Felip, E. Holgado, F. Barlesi, M. Kohlhäufl, O. Arrieta, M. A. Burgio, J. Fayette, H. Lena, E. Poddubskaya, D. E. Gerber, S. N. Gettinger, C. M. Rudin, N. Rizvi, L. Crinò, G. R. Blumenschein, S. J. Antonia, C. Dorange, C. T. Harbison, F. Graf Finckenstein, J. R. Brahmer, Nivolumab versus Docetaxel in Advanced Nonsquamous Non-Small-Cell Lung Cancer, N. Engl. J. Med. 373, 1627-1639 (2015).
63. J. Galon, B. Mlecnik, G. Bindea, H. K. Angell, A. Berger, C. Lagorce, A. Lugli, I. Zlobec, A. Hartmann, C. Bifulco, I. D. Nagtegaal, R. Palmqvist, G. V. Masucci, G. Botti, F. Tatangelo, P. Delrio, M. Maio, L. Laghi, F. Grizzi, M. Asslaber, C. D'Arrigo, F. Vidal-Vanaclocha, E. Zavadova, L. Chouchane, P. S. Ohashi, S. Hafezi-Bakhtiari, B. G. Wouters, M. Roehrl, L. Nguyen, Y. Kawakami, S. Hazama, K. Okuno, S. Ogino, P. Gibbs, P. Waring, N. Sato, T. Torigoe, K. Itoh, P. S. Patel, S. N. Shukla, Y. Wang, S. Kopetz, F. A. Sinicrope, V. Scripcariu, P. A. Ascierto, F. M. Marincola, B. A. Fox, F. Pagès, Towards the introduction of the “Immunoscore” in the classification of malignant tumours, J. Pathol. 232, 199-209 (2014).
64. K. F. Rabe, S. Hurd, A. Anzueto, P. J. Barnes, S. A. Buist, P. Calverley, Y. Fukuchi, C. Jenkins, R. Rodriguez-Roisin, C. van Weel, J. Zielinski, Global Initiative for Chronic Obstructive Lung Disease, Global strategy for the diagnosis, management, and prevention of chronic obstructive pulmonary disease: GOLD executive summary, Am. J. Respir. Crit. Care Med. 176, 532-555 (2007).
65. M.-C. Dieu-Nosjean, M. Antoine, C. Danel, D. Heudes, M. Wislez, V. Poulot, N. Rabbe, L. Laurans, E. Tartour, L. de Chaisemartin, S. Lebecque, W.-H. Fridman, J. Cadranel, Long-term survival for patients with non-small-cell lung cancer with intratumoral lymphoid structures, J. Clin. Oncol. Off. J. Am. Soc. Clin. Oncol. 26, 4410-4417 (2008).
66. B. Mlecnik, G. Bindea, H. K. Angell, P. Maby, M. Angelova, D. Tougeron, S. E. Church, L. Lafontaine, M. Fischer, T. Fredriksen, M. Sasso, A. M. Bilocq, A. Kirilovsky, A. C. Obenauf, M. Hamieh, A. Berger, P. Bruneval, J.-J. Tuech, J.-C. Sabourin, F. Le Pessot, J. Mauillon, A. Rafii, P. Laurent-Puig, M. R. Speicher, Z. Trajanoski, P. Michel, R. Sesboüe, T. Frebourg, F. Pagès, V. Valge-Archer, J.-B. Latouche, J. Galon, Integrative Analyses of Colorectal Cancer Show Immunoscore Is a Stronger Predictor of Patient Survival Than Microsatellite Instability, Immunity 44, 698-711 (2016).
67. E. D. Thompson, M. Zahurak, A. Murphy, T. Cornish, N. Cuka, E. Abdelfatah, S. Yang, M. Duncan, N. Ahuja, J. M. Taube, R. A. Anders, R. J. Kelly, Patterns of PD-L1 expression and CD8 T cell infiltration in gastric adenocarcinomas and associated immune stroma, Gut (2016), doi:10.1136/gutjnl-2015-310839.

1. A method of treating non-small cell lung cancer (NSCLC) in a patient suffering from chronic obstructive pulmonary disease (COPD) comprising administering to the patient a therapeutically effective amount of an immune checkpoint inhibitor.

2. The method of claim 1 wherein the immune checkpoint inhibitor is a TIM-3 inhibitor or is a PD-1 inhibitor.

3. The method of claim 2 wherein the immune checkpoint inhibitor is an antibody selected from the group consisting of anti-PD1 antibodies, anti-PDL1 antibodies, anti-PDL2 antibodies, anti-Galectin 9 antibodies and anti-TIM-3 antibodies.

4. The method of claim 2 wherein the immune checkpoint inhibitor is an inhibitor of TIM-3 or PD-1 expression.

5. The method of claim 2 wherein the immune checkpoint inhibitor is a multispecific antibody comprising at least one binding site that specifically binds to a PD-1 molecule, and at least one binding site that specifically binds to a TIM-3 molecule.

6. A method of modifying the activation status of lung immune cells, modulating lymphocyte distribution in the tumor microenvironment or preventing tumor-infiltrating lymphocyte (TIL) exhaustion in the tumor microenvironment in a patient suffering from a non-small cell lung cancer (NSCLC) which coexists with chronic obstructive pulmonary disease (COPD) comprising: (i) identifying the patient as a patient suffering from a non-small cell lung cancer (NSCLC) which coexists with chronic obstructive pulmonary disease (COPD); and (ii) administering to said patient a therapeutically effective amount of an immune checkpoint inhibitor.

7-9. (canceled)

10. A method of treating a patient suffering from non-small cell lung cancer (NSCLC), comprising
i) determining whether the patient has a coexisting chronic obstructive pulmonary disease (COPD) and
ii) administering an immune checkpoint inhibitor to a subject identified as having a coexisting chronic obstructive pulmonary disease.

11. A method of treating a patient suffering from a non-small cell lung cancer (NSCLC) that coexists with chronic obstructive pulmonary disease (COPD) comprising i) determining the expression level of PD-L1 in a tumor tissue sample obtained from the patient ii) comparing the expression level determined at step i) with a predetermined reference value and iii) administering to the patient a therapeutically effective amount of a PD-1 inhibitor when the level determined at step i) is higher than the predetermined reference value.

12. The method of claim 11 wherein the expression level of PD-L1 in the tumor tissue sample is determined by immunohistochemistry or is determined by determining the quantity of mRNA encoding for PD-L1.

13. The method of claim 11 which comprises determining the expression level of at least one further immune checkpoint protein.

14. A method for determining the survival time of a patient suffering from a non small cell lung cancer (NSCLC) that coexists with moderate to severe chronic obstructive pulmonary disease (COPD) and treating the patient comprising
i) providing a tumor tissue sample from the patient,
ii) quantifying the density of CD8 I cells in the tumor tissue sample,
iii) comparing the density quantified at step ii) with a predetermined reference value
iv) determining whether the tumor tissue sample is positive or negative for PD-L1 expression
wherein the patient will have a long survival time when the density quantified at step ii) is higher than its corresponding predetermined reference value and the tumor tissue sample is negative for PD-L1 expression, or
the patient will have a short survival time when the density quantified at step ii) is lower than its predetermined value independently from the fact that the tumor sample is positive or negative for PD-L1 expression or the density quantified at step ii) is higher than its corresponding predetermined reference value and the tumor tissue sample is positive for PD-L1 expression, and
v) administering a PD-1 inhibitor to a patient whose measurement is indicative of having a short survival time.

15. (canceled)

16. The method of claim 13, wherein the at least one further immune checkpoint protein is TIM-3
